(19) United States
(12) Patent Application Publication
(10) Pub. No.: US 2012/0101053 A1
(43) Pub. Date: Apr. 26, 2012

(54) CATHEPSIN CYSTEINE PROTEASE INHIBITORS FOR THE TREATMENT OF VARIOUS DISEASES

(76) Inventors: Cameron Black, Baie d' Urfe (CA); Christian Beaulieu, Laval (CA)

(21) Appl. No.: 13/379,200

(22) PCT Filed: Jun. 16, 2010
(86) PCT No.: PCT/CA10/00947

§ 371 Date: Dec. 19, 2011

Related U.S. Application Data
(60) Provisional application No. 61/219,035, filed on Jun. 22, 2009.
(60) Provisional application No. 61/299,037, filed on Jan. 28, 2010.

(51) Int. Cl. A61K 31/7048; A61K 31/4168; A61K 31/522; A61K 31/351; A61K 31/497; A61K 31/661; A61K 31/663; A61K 31/675; A61K 31/4706; A61K 31/4709; A61K 31/506; A61K 31/65; A61K 31/4355; A61K 31/366; A61K 31/29; A61K 31/4985; A61K 31/47; A61K 31/53; A61K 31/165; A61K 31/429; A61K 31/433; C07C 233/40; C07D 277/76; C07D 285/125; A61P 33/02; A61P 33/06; A61K 31/541

(52) U.S. Cl. 514/31; 514/227.8; 514/400; 514/263.3; 514/460; 514/254.07; 514/114; 514/89; 514/313; 514/314; 514/256; 514/154; 514/279; 514/450; 514/503; 514/250; 514/311; 514/242; 514/618; 514/367; 514/363; 564/162; 548/174; 548/136

(57) ABSTRACT

The present invention relates to compounds capable of inhibiting and/or decreasing the activity of one or more cathepsins, thereby treating and/or preventing various disease states associated with one or more cysteine proteases including, but not limited to, cathepsins and papain-like cysteine proteases. Disease states treated and/or prevented by the compounds of the invention include, but are not limited to, mammalian parasitic diseases in which the parasite utilizes a critical cysteine protease from the papain family. Examples of parasitic diseases to be treated or prevented by the compounds of the invention include, but are not limited to, toxoplasmosis, malaria, African trypanosomiasis, Chagas disease, leishmaniasis, coccidiosis, giardiosis, cryptosporidiosis or schistosomiasis.

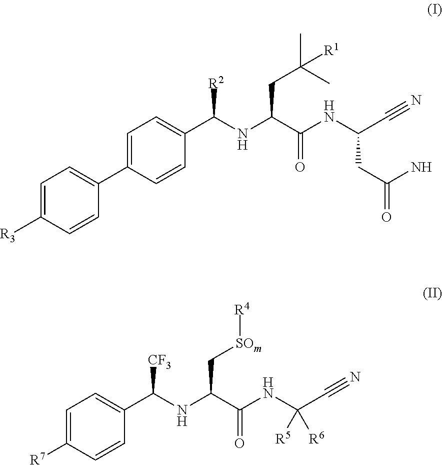

### BACKGROUND OF THE INVENTION

Cathepsins belong to the papain superfamily of cysteine proteases. These proteases function in the normal physiological as well as pathological degradation of connective tissue. Cathepsins play a major role in intracellular protein degradation and turnover and remodeling. To date, a number of cathepsin have been identified and sequenced from a number of sources. These cathepsins are naturally found in a wide variety of tissues. For example, cathepsin B, C, F, H, L, K, O, S, V, W, and Z have been cloned. Cathepsin K (which is also known by the abbreviation cat K) is also known as cathepsin O and cathepsin O2. See PCT Application WO 96/13523, Khepri Pharmaceuticals, Inc., published May 9, 1996, which is hereby incorporated by reference in its entirety. Cathepsin K is implicated in a variety of disease states which include, but are not limited to, osteoporosis, glucocorticoid induced osteoporosis, Paget's disease, abnormally increased bone turnover, periodontal disease, tooth loss, bone fractures, atherosclerosis, obesity, rheumatoid arthritis, osteoarthritis, periprosthetic osteolysis, osteogenesis imperfecta, metastatic bone disease, hypercalcemia of malignancy, and multiple myeloma. Cathepsin L is implicated in normal lysosomal proteolysis as well as several diseases states, including, but not limited to, metastasis of melanomas. Cathepsin S is implicated in Alzheimer's disease, asthma, atherosclerosis, chronic obstructive pulmonary disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis; allergic disorders, including, but not limited to asthma; and allogenic immune responses, including, but not limited to, rejection of organ transplants or tissue grafts. Increased Cathepsin B levels and redistribution of the enzyme are found in tumors, suggesting a role in tumor invasion and metastasis. In addition, aberrant Cathepsin B activity is implicated in such disease states as rheumatoid arthritis, osteoarthritis, pneumocystisis carinii, acute pancreatitis, inflammatory airway disease and bone and joint disorders.

Mammalian cathepsins are related to the papain-like cysteine proteases expressed by disease-causing parasites including those from the families protozoa, platyhelminthes, nematodes and arthropodes. These cysteine proteases play an essential role in the life cycle of these organisms.

Several parasites responsible for mammalian diseases are dependent on cysteine protease for various life-cycle functions. Inhibition of these proteases can be useful in the treatment of these parasitic diseases, see Lecaille, F., et al, Chem. Rev., 102, 4459-4488, 2002.

Cruzipain is a cysteine protease enzyme present in Trypanosoma cruzi and is thought to play an important role in all stages of the parasite's life cycle. The enzyme is highly expressed in the epimastigote stage where it is primarily a lysosomal enzyme and may be involved in protein digestion during differentiation to the infective metacyclic trypomastigote stage. Identification of cruzipain in the membrane of the trypomastigote implicates this enzyme in the penetration of the parasite into the host cell. Cruzipain is also found in the membranes of the amastigote form of the parasite, see Cazzulo, J. J., et al, Current Pharmaceutical Design, 7, 1143-1156, 2001. Cruzipain efficiently degrades human IgG, which may play a protective role for the parasite by preventing antigen presentation and thus reducing the host immune response. Based on these observations, it has been proposed that cruzipain is a valid drug target for chemotherapy of Chagas disease. Cruzipain has been reported to exist in at least two polymorphic sequences, known as cruzipain 1 and cruzipain 2, both of which may be involved in the viability of Trypanosoma cruzi (Lima, et al, Molecular & Parasitology 114, 41-52, 2001).

A similar role for the cysteine protease trypanopain-Tb has been proposed in the life-cycle of Trypanosoma brucei, the parasite responsible for African trypanosomaisis, or sleeping sickness.

A similar parasite, T. congolense, is responsible for the bovine disease trypanosomiasis. Congopain is the analogous cysteine protease to cruzipain in this parasite.

Falcipain is an important cysteine protease in Plasmodium falicparum. This enzyme is reported to be important in the degradation of host hemoglobin in parasite food vacuoles. The processing of hemoglobin is essential to the growth of the parasite, thus an inhibitor of falcipain should be useful as a treatment for malaria.

Two cysteine proteases, SmCL1 and SmCL2, are present in the human blood fluke Schistosoma mansoni. SmCL1 may play a role in the degradation of host hemoglobin, while SmCL2 may be important to the reproductive system of the parasite (Brady, C. P., et al, Archives of Biochemistry and Biophysics, 380, 46-55, 2000). Inhibition of one or both of these proteases may provide an effective treatment for human schistosomiasis.

LmajcatB and CP2.8ΔCTE are important cysteine proteases of the parasitic protazoa Leishmania major and Leishmania mexicanus respectively, see Alves, L. C., et al, Eur. J. Biochem, 268, 1206-1212, 2001 Inhibition of these enzymes may provide a useful treatment for leishmaniasis.

Giardia lamblia also contains an essential cysteine protease that may be inhibited by compounds of this invention. See DuBois et al, JBC 283, 18024-18031, 2008.

Cryptosporidium parvum also contains an essential cysteine protease, cryptopain 1, that may be inhibited by compounds of this invention. See Na et al, Parasitology. 136, 149-157, 2009.

Parasites of the Eimeria family, may also be susceptible to compounds of the present invention, allowing for treatment of coccidiosis.

The present invention relates to compounds that are capable of inhibiting cathepsins, thereby treating and preventing various disease states, including mammalian parasitic diseases in which the parasite utilizes a critical cysteine protease from the papain family.

### SUMMARY OF THE INVENTION

The present invention relates to compounds capable of inhibiting and/or decreasing the activity of one or more cathepsins, thereby treating and/or preventing various disease states associated with one or more cysteine proteases including, but not limited to, cathepsins and papain-like cysteine proteases.

One embodiment of the present invention is illustrated by a compound of Formula I, and the pharmaceutically acceptable salts and stereoisomers thereof:

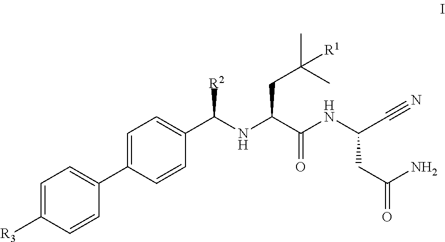

Another embodiment of the present invention is illustrated by a compound of Formula II, and the pharmaceutically acceptable salts and stereoisomers thereof:

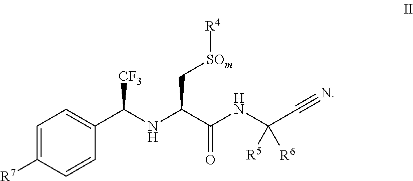

### DETAILED DESCRIPTION OF THE INVENTION

The present invention relates to compounds capable of inhibiting and/or decreasing the activity of one or more cathepsins, thereby treating and/or preventing various disease states associated with one or more cysteine proteases including, but not limited to, cathepsins and papain-like cysteine proteases. Disease states treated and/or prevented by the compounds of the invention include, but are not limited to, mammalian parasitic diseases in which the parasite utilizes a critical cysteine protease from the papain family (e.g., toxoplasmosis, malaria, African trypanosomiasis, Chagas disease, leishmaniasis, coccidiosis, giardiosis, cryptosporidiosis or schistosomiasis), osteoporosis, glucocorticoid induced osteoporosis, Paget's disease, abnormally increased bone turnover, periodontal disease, tooth loss, bone fractures, atherosclerosis, obesity, rheumatoid arthritis, osteoarthritis, periprosthetic osteolysis, osteogenesis imperfecta, metastatic bone disease, hypercalcemia of malignancy, multiple myeloma, metastasis of melanomas, Alzheimer's disease, asthma, chronic obstructive pulmonary disease, juvenile onset diabetes, multiple sclerosis, pemphigus vulgaris, Graves' disease, myasthenia gravis, systemic lupus erythemotasus, Hashimoto's thyroiditis, allogenic immune responses, pneumocystisis carinii, acute pancreatitis, inflammatory airway disease and bone and joint disorders.

One embodiment of the present invention is illustrated by a compound of the following formula I, and the pharmaceutically acceptable salts and stereoisomers thereof:

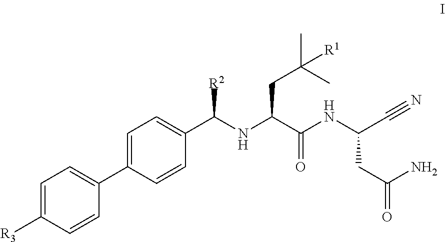

wherein R¹ is hydrogen or halo;
R² is C₁₋₃ alkyl which is substituted with two to seven halo;
R³ is hydrogen, C₁₋₆ alkyl, halo or —SOₘ(C₁₋₆ alkyl);
m is an integer from zero to two;
or a pharmaceutically acceptable salt thereof.

In a class of the invention, R¹ is hydrogen.

In a class of the invention, R² is trifluoromethyl.

Another embodiment of the present invention is illustrated by a compound of the following formula II, and the pharmaceutically acceptable salts and stereoisomers thereof:

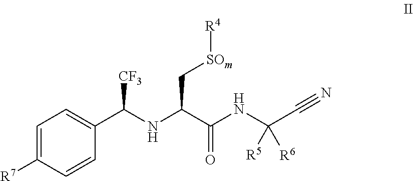

Wherein R⁴ is C₁₋₆alkyl, C₁₋₆ haloalkyl, C₃₋₆cycloalkyl, aryl, (C₁₋₆alkyl)aryl, heteroaryl, or (C₁₋₆alkyl)heteroaryl, wherein said aryl and heteroaryl groups are optionally substituted with 1 to 3 substituents independently selected from the group consisting of C₁₋₆alkyl, halo, C₁₋₆ haloalkyl and cyano;

R⁵ is hydrogen, C₁₋₆ alkyl or benzyl, wherein said alkyl group is optionally substituted with 1 to 6 halo and said benzyl group is optionally substituted with one to three groups independently selected from the group consisting of halo, cyano, hydroxyl, C₁₋₆ alkyl and SOₘ;
R⁶ is hydrogen or C₁₋₆ alkyl, wherein said alkyl group is optionally substituted with 1 to 6 halo; or R⁵ and R⁶, together with the carbon atom to which they are attached, form a C₃₋₈ cycloalkyl ring which is optionally substituted with C₁₋₆ alkyl or halo;
R⁷ is hydrogen, halo, C₁₋₆ alkyl, C₁₋₆ haloalkyl, C₃₋₆cycloalkyl, aryl or heteroaryl wherein said aryl and heteroaryl groups are optionally substituted with 1 to 3 substituents independently selected from the group consisting of halo, hydroxyl, C₁₋₆alkyl, C₁₋₆ haloalkyl and cyano;
m is an integer from zero to two.

In a class of the invention, R⁴ is C₁₋₆alkyl. In another class of the invention, R⁴ is (C₁₋₆alkyl)aryl, wherein said aryl group is optionally substituted with 1 to 3 halo.

In a class of the invention, R⁵ is hydrogen and R⁶ is hydrogen. In another class of the invention, R⁵ and R⁶, together with the carbon atom to which they are attached, form a cyclopropyl ring.

In a class of the invention, R⁷ is hydrogen or halo.

Reference to the preferred embodiments set forth above is meant to include all combinations of particular and preferred groups unless stated otherwise.

Specific embodiments of the papain family cysteine protease inhibitors of the present invention include, but are not limited to:

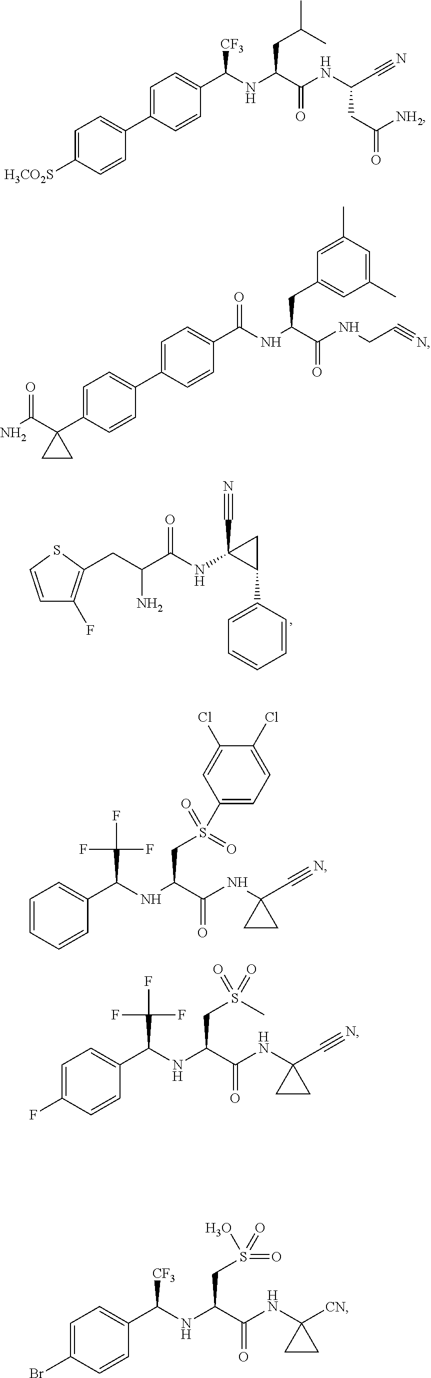

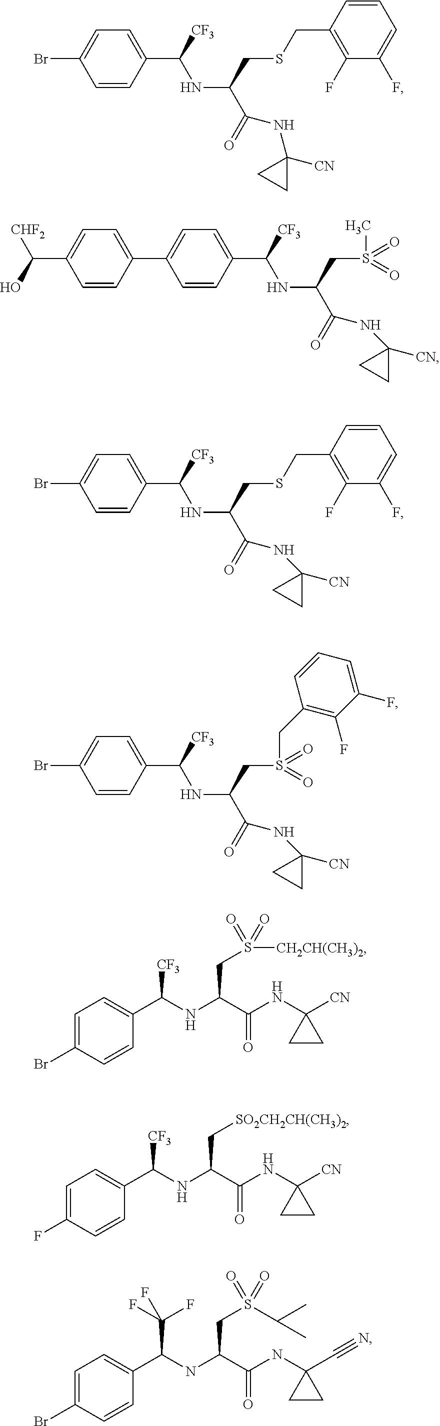

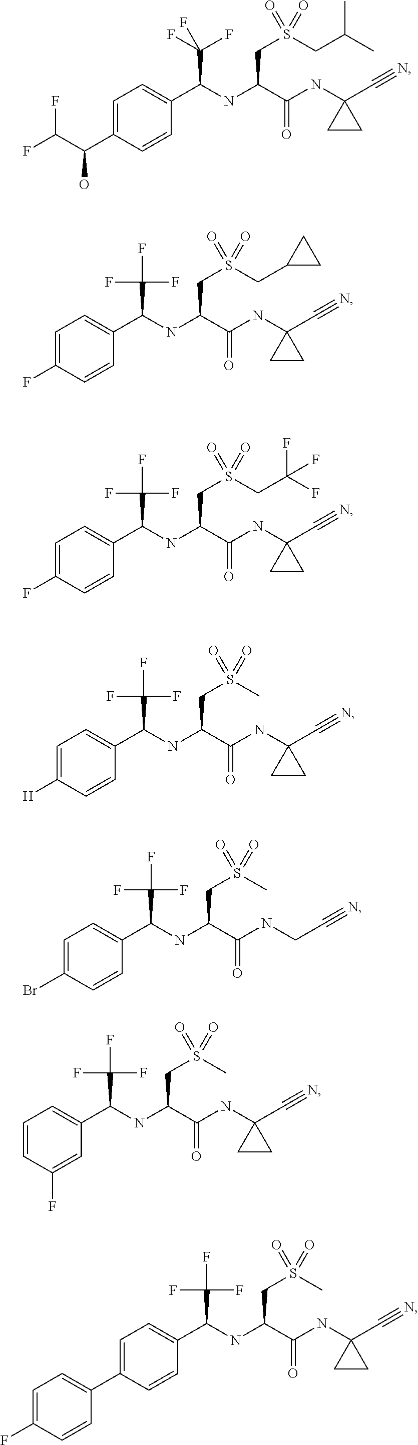

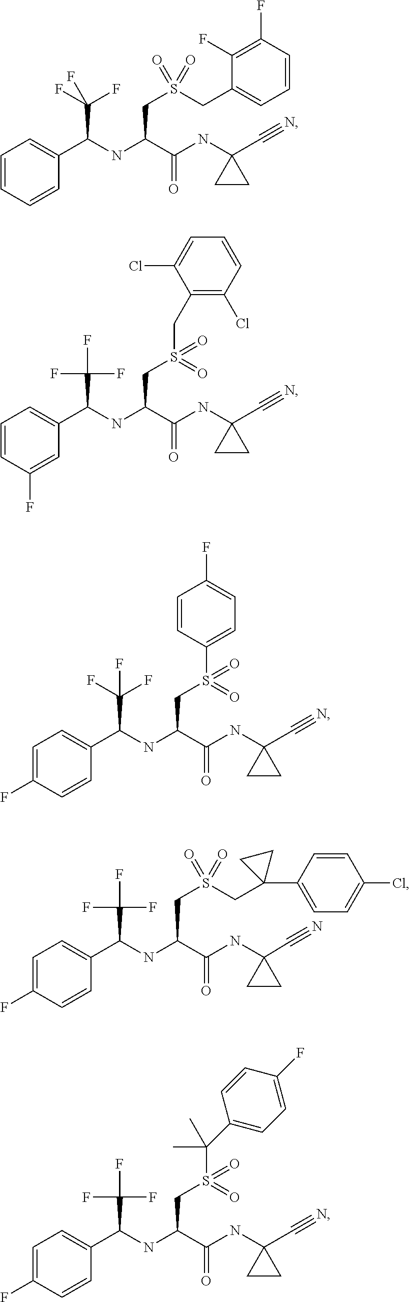

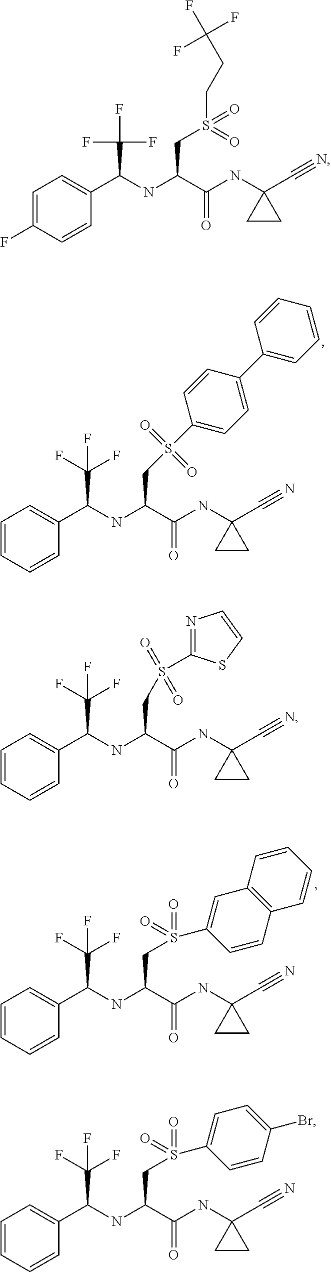

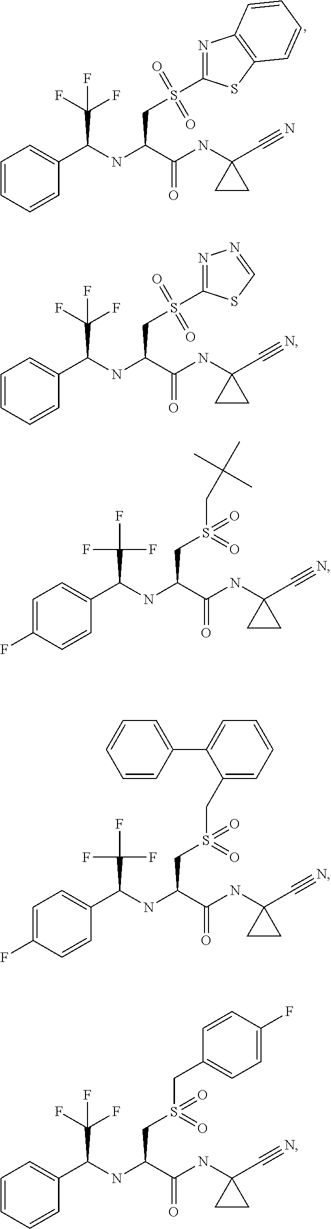

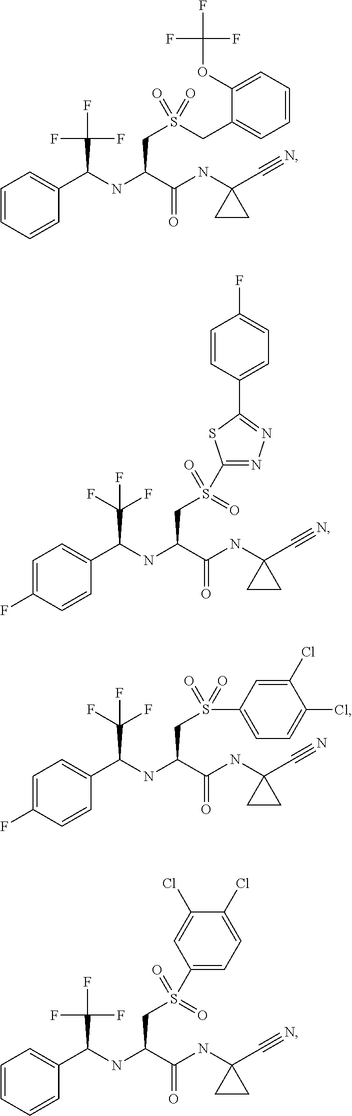

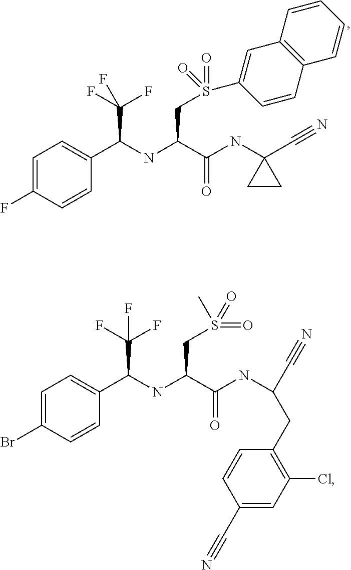

or a pharmaceutically acceptable salt or stereoisomer thereof.

Also included within the scope of the present invention is a pharmaceutical composition which is comprised of a compound as described above and a pharmaceutically acceptable carrier. The invention is also contemplated to encompass a pharmaceutical composition which is comprised of a pharmaceutically acceptable carrier and any of the compounds specifically disclosed in the present application. The invention also encompasses the use of the compounds of the invention for the preparation of a medicament for the treatment and/or prevention of cysteine protease-related diseases or conditions. These and other aspects of the invention will be apparent from the teachings contained herein.

### Utilities

The compounds of the present invention inhibit and/or decrease the activity of cathepsins and are therefore useful to treat and/or prevent cathepsin dependent diseases or conditions in mammals, preferably humans. In specific embodiments, the compounds of the present invention inhibit and/or decrease the activity of Cathepsins K, B, L, S, and papain-like cysteine proteases expressed by disease-causing parasites.

“Cathepsin dependent disease or condition” refers to a pathologic condition that depends on the activity, either in whole or in part, of one or more cathepsins. “Cathepsin K dependent disease or condition” refers to a pathologic condition that depends, either in whole or in part, on the activity of Cathepsin K. Diseases associated with Cathepsin K activities include, but are not limited to, osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, atherosclerosis and cancer including metastatic bone disease, hypercalcemia of malignancy, and multiple myeloma. In treating such conditions with the instantly claimed compounds, the required therapeutic amount will vary according to the specific disease and is readily ascertainable by those skilled in the art. Although both treatment and prevention are contemplated by the scope of the invention, the treatment of these conditions is the preferred use.

“Cathepsin L dependent disease or condition” refers to a pathologic condition that depends, either in whole or in part, on the activity of Cathepsin L. Diseases associated with Cathepsin L activities include, but are not; limited to, metastasis of melanomas.

“Cathepsin S dependent disease or condition” refers to a pathologic condition that depends, either in whole or in part, on the activity of Cathepsin S. Diseases associated with Cathepsin S activities include, but are not limited to, Alzheimer's disease, asthma, atherosclerosis, chronic obstructive pulmonary disease and certain autoimmune disorders, (e.g., juvenile onset diabetes, multiple sclerosis, pemphigus vulgaris, Graves' disease, myasthenia gravis, systemic lupus erythemotasus, rheumatoid arthritis and Hashimoto's thyroiditis); allergic disorders (e.g., asthma); and allogenic immune responses (e.g., rejection of organ transplants or tissue grafts).

“Cathepsin B dependent disease or condition” refers to a pathologic condition that depends, either in whole or in part, on the activity of Cathepsin B. Diseases associated with Cathepsin B activities include, but are not limited to, tumor invasion and metastasis, rheumatoid arthritis, osteoarthritis, pneumocystisis carinii, acute pancreatitis, inflammatory airway disease and bone and joint disorders.

“Cysteine protease-related disease or condition” refers to a pathologic condition that depends, either in whole or in part, on the activity of one or more cysteine proteases, especially papain-like cysteine proteases. Diseases associated with cysteine proteases include diseases associated with the activities of Cathepsin K, Cathepsin L, Cathepsin S, Cathepsin B and parasites that are dependent on cysteine proteases for various life-cycle functions.

Mammalian cathepsins are related to the papain-like cysteine proteases expressed by disease-causing parasites including those from the families protozoa, platyhelminthes, nematodes and arthropodes. These cysteine proteases play an essential role in the life cycle of these organisms.

The use of cysteine protease inhibitors for the treatment of Chagas disease and African trypanosomaisis has been discussed in the art. Substantiation of this hypothesis has been provided by the observation that irreversible inhibitors of cruzipain can cure Chagas disease in mouse models, see Engel, J., et al, J. Exp. Chem., 188, 725-734, 1998. Cruzipain has been reported to exist in at least two polymorphic sequences, known as cruzipain 1 and cruzipain 2, both of which may be involved in the viability of Trypanosoma cruzi (Lima, et al, Molecular & Parasitology 114, 41-52, 2001). A similar role for the cysteine protease trypanopain-Tb has been proposed in the life-cycle of Trypanosoma brucei, the parasite responsible for African trypanosomaisis, or sleeping sickness.

The use of cysteine protease inhibitors for the treatment of malaria has been discussed in the art. Anti-malarial activity has been found with irreversible falcipain inhibitors in a mouse model of malaria (P. vinckei infection), see Olson, J. E., et al, Biorg. Med. Chem., 7, 633-638, 1999.

Two cysteine proteases, SmCL1 and SmCL2, are present in the human blood fluke Schistosoma mansoni. SmCL1 may play a role in the degradation of host hemoglobin, while SmCL2 may be important to the reproductive system of the parasite, see Brady, C. P., et al, Archives of Biochemistry and Biophysics, 380, 46-55, 2000. Thus, inhibition of one or both of these proteases may provide an effective treatment for human schistosomiasis.

LmajcatB and CP2.8ΔCTE are important cysteine proteases of the parasitic protazoa Leishmania major and Leishmania mexicanus respectively, see Alves, L. C., et al, Eur. J. Biochem, 268, 1206-1212, 2001. Thus, inhibition of these enzymes may provide a useful treatment for leishmaniasis.

Exemplifying the invention is the use of any of the compounds described above in the preparation of a medicament for the treatment and/or prevention of Chagas disease, toxoplasmosis, malaria, African trypanosomiasis, leishmaniasis coccidiosis, giardiosis, cryptosporidiosis or schistosomiasis in a mammal in need thereof. The compounds of this invention may be administered to mammals, preferably humans, either alone or preferably in combination with pharmaceutically acceptable carriers or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and topical routes of administration.

In the case of tablets for oral use, carriers which are commonly used include lactose and corn starch, and lubricating agents, such as magnesium stearate, are commonly added. For oral administration in capsule form, useful diluents include lactose and dried corn starch. For oral use of a therapeutic compound according to this invention, the selected compound may be administered, for example, in the form of tablets or capsules, or as an aqueous solution or suspension. For oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The instant compounds are also useful in combination with known agents useful for treating and/or preventing cysteine protease-related diseases or conditions such as parasitic diseases, including toxoplasmosis, malaria, African trypanosomiasis, Chagas disease, leishmaniasis or schistosomiasis. Combinations of the presently disclosed compounds with other agents useful in treating and/or preventing cysteine protease-related diseases or conditions such as parasitic diseases are within the scope of the invention. A person of ordinary skill in the art would be able to discern which combinations of agents would be useful based on the particular characteristics of the drugs and the disease involved.

Existing therapies for Chagas Disease include, but are not limited to nifurtimox, benznidazole, and allopurinol. Drugs that may have an effect on the parasite include but are not limited to terbinafine, lovastatin, ketoconazole, itraconazole, posaconazole, miltefosine, ilmofosine, pamidronate, alendronate, and risedronate. Other mechanisms being explored for the treatment of Chagas Disease include, but are not limited to inhibitors of trypanothione reductase and inhibitors of hypoxanthine-guanine phosphoribosyl transferase (HGPRT), See, Urbina, Current Pharmaceutical Design, 8, 287-295, 2002)

Existing therapies for malaria include, but are not limited to chloroquine, proguanil, mefloquine, quinine, pyrimethamine-sulphadoxine, doxocycline, berberine, halofantrine, primaquine, atovaquone, pyrimethamine-dapsone, artemisinin and quinhaosu.

Existing therapies for leishmaniasis include, but are not limited to meglumine antimonite, sodium stibogluconate and amphotericin B.

Existing therapies for schistosomiasis include, but are not limited to praziquantel and oxamniquine.

Existing therapies for African trypanosomiasis include, but are not limited to pentamidine, melarsoprol, suramin and eflornithine.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents. The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985, which is incorporated by reference herein in its entirety. Metabolites of these compounds include active species produced upon introduction of compounds of this invention into the biological milieu.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes arresting or reducing the development of the disease and/or its clinical symptoms; or relieving the disease, i.e., causing regression of the disease and/or its clinical symptoms.

The terms “prevent” or “preventing” a disease as used herein includes causing the clinical symptoms of the disease not to develop in a mammal that has been or may be exposed to the causative agent of the disease or is predisposed to the disease but does not yet experience or display symptoms of the disease.

The present invention also encompasses a pharmaceutical composition useful in the treatment of parasitic diseases, comprising the administration of a therapeutically effective amount of the compounds of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for a parasitic disease. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, 200, 250, 300, 400 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The compounds of the present invention can be used in combination with other agents useful for treating parasitic diseases. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly.

These and other aspects of the invention will be apparent from the teachings contained herein.

### DEFINITIONS

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted.

When any variable (e.g. R¹, R²) occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring carbon atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having the specified number of carbon atoms. For example, C₁₋₆, as in “C₁₋₆ alkyl” is defined to include groups having 1, 2, 3, 4, 5 or 6 carbons in a linear, branched, or cyclic arrangement. For example, “C₁₋₆ alkyl” specifically includes methyl, ethyl, propyl, butyl, pentyl, hexyl, and so on.

The term “haloalkyl” means an alkyl radical as defined above, unless otherwise specified, that is substituted with one to five, preferably one to three halogens. Representative examples include, but are not limited to trifluoromethyl, dichloroethyl, and the like.

The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on.

The term “aryl” is intended to mean any stable monocyclic or bicyclic carbon ring of up to 12 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl,” as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. “Heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively. Such heteraoaryl moieties for substituent Q include but are not limited to: 2-benzimidazolyl, 2-quinolinyl, 3-quinolinyl, 4-quinolinyl, 1-isoquinolinyl, 3-isoquinolinyl and 4-isoquinolinyl.

As appreciated by those of skill in the art, “halo” or “halogen” as used herein is intended to include chloro, fluoro, bromo and iodo. The term “keto” means carbonyl (C═O).

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. Also when compounds of Formula I contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

The alkyl substituents may be unsubstituted or unsubstituted, unless specifically defined otherwise. For example, a (C₁-C₆)alkyl may be substituted with one or more substituents selected from OH, oxo, halogen, alkoxy, dialkylamino, or heterocyclyl, such as morpholinyl, piperidinyl, and so on. In the case of a disubstituted alkyl, for instance, wherein the substituents are oxo and OH, the following are included in the definition: —(C═O)CH₂CH(OH)CH₃, —(C═O)OH, —CH₂(OH)CH₂CH(O), and so on.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm. Sci., 1977:66:1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

For purposes of this specification, the following abbreviations have the indicated meanings:
CH₂Cl₂=methylene chloride
Et₃N=triethylamine
HATU=o-(7-azabenzotriazol-1-yl)-N,N,N′,N′-tetramethyluronium hexafluorophosphate
K₂CO₃=potassium carbonate
KOCH₃=potassium methoxide
MeOH=methanol
NaBH₄=sodium borohydride
PdCl₂(dppf)=[1,1′-bis(diphenylphosphino)ferrocene]dichloropalladium(II)
rt=room temperature
sat. aq.=saturated aqueous
ZnCl₂=zinc chloride
Zn(BH₄)₂=zinc borohydride
tlc=thin layer chromatography
Me=methyl
Et=ethyl
n-Pr=normal propyl
i-Pr=isopropyl

The compounds of the present invention can be prepared according to the following general procedures using appropriate materials and are further exemplified by the following specific examples. The compounds illustrated in the examples are not, however, to be construed as forming the only genus that is considered as the invention. The following examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

### Schemes

In Scheme 1, a suitably functionalized cysteine derivative 1 and a trifluoromethyl ketone 2 are treated with a base such as potassium methoxide in a solvent such as methanol. The product is treated in situ at low temperature (−40° C.) with a reducing agent such as zinc borohydride to yield the carboxylic acid 3. This acid is then coupled to the aminoacetonitrile derivative 4 with a coupling agent such as HATU and a base such as DIPEA. The sulfur may be oxidized to the corresponding sulfone using an oxidizing agent such as m-chloroperoxybenzoic acid in dichloromethane, magnesium monoperoxyphthalate in methanol or hydrogen peroxide in combination with sodium tungstate and a phase transfer reagent such as tetrabutylammonium hydrogen sulfate in ethyl acetate.

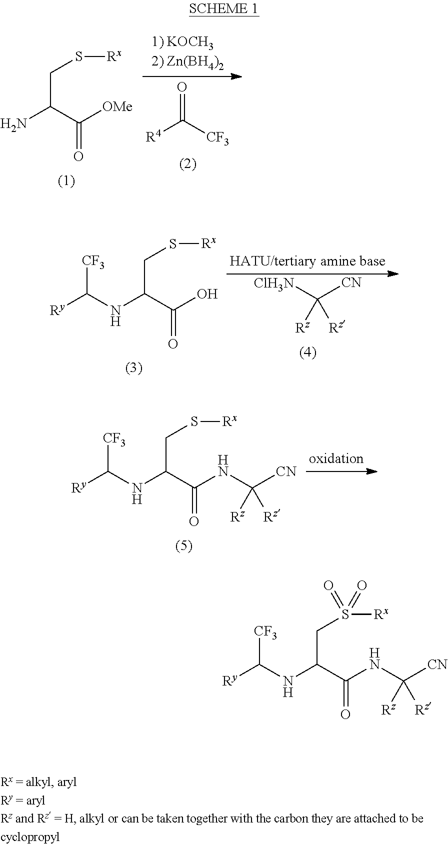

Compounds of the present invention may be prepared according to Scheme 2, as indicated below. An amino ester is reacted with an appropriately substituted ketone under basic conditions to form an imine which is further reduced with an agent such as NaBH₄ or Zn(BH₄)₂. The resulting carboxylic acid can be coupled to a substituted aminonitrile using different coupling agents such as PyBOP, HBTU or HATU. A Suzuki coupling provided the compound of the invention.

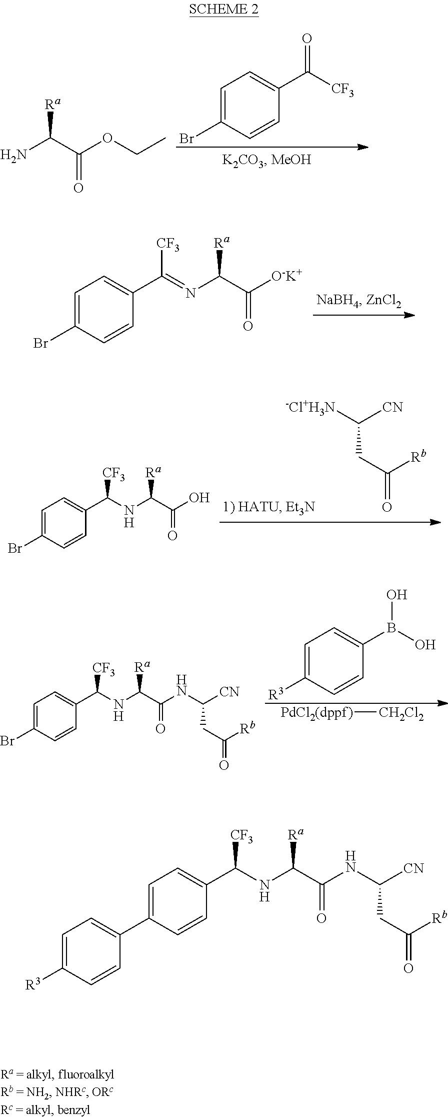

### EXAMPLES

### Example 1

### N²-[(1S)-1-(4-bromophenyl)-2,2,2-trifluoroethyl]-N¹-(1-cyanocyclopropyl)-3-(methylsulfonyl)-L-alaninamide

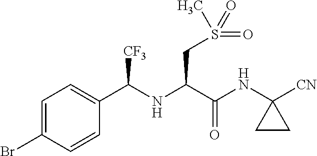

Step 1. To a −5° C. solution of methyl N-(tert-butoxycarbonyl)-L-cysteinate (45.8 g, 195 mmol) in DMF (600 mL, 0.325M) was added iodomethane (13.38 mL, 214 mmol, 1.1 eq) followed by potassium carbonate (27 g, 195 mmol, 1 eq) and the mixture was stirred overnight at 5° C. It was poured over water and little aqueous NH₄Cl and extracted with Et₂O (3×) washed with dilute NaHCO₃ and brine, dried and stripped to dryness to yield methyl N-(tert-butoxycarbonyl)-S-methyl-L-cysteinate (48.1 g), which was used as such in the next step.
Step 2. Acetyl chloride (13.72 mL, 193 mmol, 1 eq) was added slowly to −15° C. methanol (50 mL, 3.86M) and the mixture was reacted for 1 hr. The product of Step 1 (48.1 g, 193 mmol), as a methanol (20 mL) solution, was then added and the mixture was reacted at 20° C. for 4 hrs. It was evaporated to dryness and swished in MTBE (500 mL) at 20° C., then dried on high vacuum overnight to give methyl S-methyl-L-cysteinate HCl. ¹H NMR (CD₃OD) δ: 4.3-4.4 (1H, m), 3.9 (3H, s), 3.0-3.2 (2H, m), 2.2 (3H, s).
Step 3: To a −78° C. suspension of 1-(4-bromophenyl)-2,2,2-trifluoroethanone (17.5 g, 69.2 mmol) and methyl S-methyl-L-cysteinate HCl (14.85 g, 80 mmol, 1.15 eq) in MeOH (75 mL) was added potassium methoxide, 95% (10.2 g, 138 mmol, 2 eq) and the mixture was allowed to warm to RT and was stirred overnight. To this suspension, cooled to −40° C., was added CH₃CN (400 mL) and then a zinc borohydride suspension (prepared by adding sodium borohydride (10.48 g, 277 mmol, 4 eq) portion-wise to a 0° C. suspension of zinc chloride (18.81 g, 138 mmol, 2 eq) in glyme (150 mL) and stirring overnight at RT) was transferred slowly, over 30 min. at −40° C. The mixture was stirred for 2 hrs and then acetone (150 mL) was added dropwise. The mixture was then allowed to warm to RT. It was poured on ice, water and EtOAc and the pH adjusted to c.a. 5 with 1N HCl. It was extracted twice with EtOAc, washed with brine and dried. The mixture was purified on SiO2 using 1:3 EtOAc and hexanes followed by 1:3 EtOAc and hexanes containing 10% acetic acid to yield the N-[(1S)-1-(4-bromophenyl)-2,2,2-trifluoroethyl]-S-methyl-L-cysteine (12.4 g) as a mixture of isomers, which was used as such in Step 4. ¹H NMR (CD₃OD) δ: 7.6-7.7 (2H, d), 7.5 (2H, d), 4.55-4.65 (1H, m), 4.6-4.7 (1H, m), 2.85-2.95 (2H, m), 2.15 (3H, s).
Step 4. To a −5° C. solution of the mixture from Step 3 (12.4 g, 33.3 mmol), HATU (18.97 g, 49.9 mmol, 1.5 eq) and 1-amino-1-cyclopropanecarbonitrile-HCl (5.92 g, 49.9 mmol, 1.5 eq) in DMF (50 mL, 0.666M) was added DIPEA (34.9 mL, 200 mmol, 6 eq) dropwise and the mixture was reacted at 0° C. for 1.5 hr. It was poured in ice and water and then extracted twice with 1:1 EtOAc and diethyl ether. The combined organic layers were washed with water, brine and dried.

Chromatography on SiO2 using 1:3 EtOAc and hexanes followed by 1:2 EtOAc and hexanes yielded N²-[(1S)-1-(4-bromophenyl)-2,2,2-trifluoroethyl]-N¹-(1-cyanocyclopropyl)-S-methyl-L-cysteinamide (11.2 g), which again contained an isomer and was used as such in the next step.

MS (+ESI): 371.8, 373.8 [M+1]⁺.

Step 5. To a 0° C. suspension of the mixture from Step 4 (11.2 g, 25.7 mmol) in EtOAc (250 mL, 0.103M) was added sodium tungstate dihydrate (102 mg, 0.308 mmol, 0.012 eq) and tetrabutyl-ammonium hydrogen sulfate (445 mg, 1.311 mmol, 0.051 eq). To this was added hydrogen peroxide 30% (6.43 mL, 62.9 mmol, 2.449 eq) dropwise and the mixture was allowed to warm to RT and was stirred for 4 hrs. The mixture was diluted with EtOAc and washed with dilute aqueous sodium thiosulfate and brine. It was purified by chromatography on SiO₂ using 1:25 MeOH:CH₂Cl₂. The residue after evaporation was triturated in MTBE for 16 hours. Filtration and drying yielded the title compound (9.6 g). ¹H NMR (CD₃COCD₃) δ 8.5 (1H, bs), 7.6-7.7 (2H, d), 7.4-7.5 (2H, d), 4.45-4.55 (1H, m), 3.75-3.75 (1H, m), 3.5-3.6 (1H, m), 3.25-3.4 (2H, m), 3.15 (3H, s), 1.4-1.5 (2H, m), 1.05-1.25 (2H, m).

### Example 2

### N²-[(1S)-1-(4-bromophenyl)-2,2,2-trifluoroethyl]-N¹-(1-cyanocyclopropyl)-S-(2,3-difluorobenzyl)-L-cysteinamide

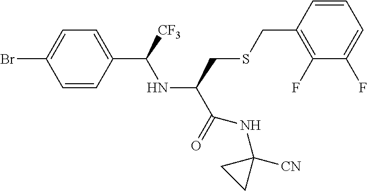

Step 1. Following the procedure of Example 1, Step 1 using 1-(bromomethyl)-2,3-difluorobenzene, methyl N-(tert-butoxycarbonyl)-S-(2,3-difluorobenzyl)-L-cysteinate was prepared and used as such in the next step.
Step 2. Following the procedure of Example 1, Step 2, using the compound of above Step 1 methyl S-(2,3-difluorobenzyl)-L-cysteinate HCl was prepared. ¹H NMR (CD₃OD) δ: 7.1-7.35 (3H, m), 4.35 (1H, m), 3.8-4.0 (5H, m), 2.95-3.13 (2H, m).
Step 3. Following the procedure of Example 1, Step 3 using the compound of above Step 2, N-[(1S)-1-(4-bromophenyl)-2,2,2-trifluoroethyl]-S-(2,3-difluorobenzyl)-L-cysteine was obtained as an impure mixture and was used as such in the next step.
Step 4. To a −5° C. solution of the compound of Step 3 (2.09 g, 4.32 mmol), HATU (2.464 g, 6.48 mmol, 1.5 eq) and 1-Amino-1-cyclopropanecarbonitrile-HCl (768 mg, 6.48 mmol, 1.5 eq) in DMF (10 mL, 0.432M) was added DIPEA (4.52 mL, 25.9 mmol, 6 eq) dropwise and the mixture was reacted at R.T. for 3 hrs. It was poured on ice and dilute HCl and extracted twice with EtOAc. The combined organic layers were washed with dilute NaHCO₃, brine, dried and the solvent was removed in vacuo. The residue was chromatographed on SiO2 using 1:2 EtOAc and hexanes to yield the title compound (1.4 g) as a mixture of isomers with about 10% of Preparation of L-001484013, N²-[(1R)-1-(4-bromophenyl)-2,2,2-trifluoroethyl]-N¹-(1-cyano-cyclopropyl)-S-(2,3-difluorobenzyl)-L-cysteinamide; this product was used as such in Example 4. MS (+ESI): 548.3, 550.2.

### Example 3

### N¹-(1-Cyanocyclopropyl)-3-(methylsulfonyl)-N²-[(1S)-2,2,2-trifluoro-1-(4-fluorophenyl)ethyl]-L-alaninamide

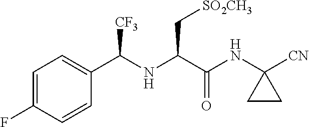

Step 1. Following the procedure of Example 1, Step 3, but using 2,2,2,4% tetrafluoroacetophenone and methyl S-methyl-L-cysteinate HCl was prepared S-methyl-N-[(1S)-2,2,2-trifluoro-1-(4-fluorophenyl)ethyl]-L-cysteine which was used as such in Step 2. ¹H NMR (CD₃COCD₃) δ: 7.55-7.65 (2H, m), 7.15-7.25 (2H, m), 4.55-4.65 (1H, m), 3.6-3.7 (1H, m), 2.8-3.0 (3H, hidden under water), 2.15 (3H, s).
Step 2. Following the procedure of Example 1, Step 4, using the compound of above Step 1 N¹-(1-cyanocyclopropyl)-S-methyl-N²-[(1S)-2,2,2-trifluoro-1-(4-fluorophenyl)ethyl]-L-cysteinamide was prepared. ¹H NMR (CD₃COCD₃) δ: 8.3 (1H, NH), 7.55-7.65 (2H, m), 7.25-7.35 (2H, m), 4.5 (1H, m), 3.35-3.45 (1H, m), 3.0 (1H, NH), 2.8-2.9 (2H, m), 2.1 (3H, s), 1.35-1.45 (2H, m), 1.0-1.2 (2H, m).
Step 3. Following the procedure of Example 1, Step 5 using the compound of above Step 2, the title compound was prepared. MS (+ESI): 407.9 [M+1]⁺.

### Example 4

### N²-[(1S)-1-phenyl-2,2,2-trifluoroethyl]-N¹-(1-cyanocyclopropyl)-3-[(3,4-dichlorophenyl)sulfonyl]-L-alaninamide

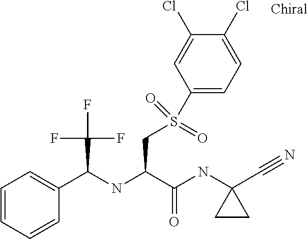

Using the methods described above, starting with methyl S-(3,4-dichlorophenyl)-L-cysteinate HCl and 2,2,2-trifluoroacetophenone, the title compound was prepared.

M+H (+ESI)=521.4

### Example 5

### (2S)-2-amino-N-[(1R,2R)-1-cyano-2-phenylcyclopropyl]-3-(3-fluoro-2-thienylpropanamide

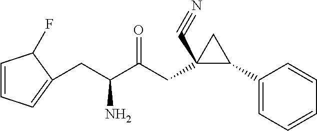

Step 1: Synthesis of 3-fluoro-2-thiophenecarboxylic acid

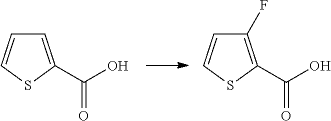

To a solution of 2-thiophenecarboxylic acid (1 eq) in THF (0.4M) at −78° C. was added dropwise n-BuLi (2.2 eq). The mixture was stirred at −78° C. for 30 min and N-Fluorobenzenesulfonimide in THF (0.5M) was added. The resulting mixture was stirred at −78° C. for 3 h and warmed to rt overnight. The reaction mixture was then cooled to 5° C. and diluted with diethyl ether and 1N aqueous HCl. Organic and aqueous layers were separated and the aqueous layer was extracted with diethyl ether (3×). The combined organic layers were dried over magnesium sulfate, filtered and concentrated. The desired product was purified by Combiflash purification on silica gel using a gradient of 20-50% EtOAc/Hexanes.

Step 2: Synthesis of (3-fluoro-2-thienyl)methanol

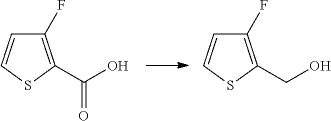

To a solution of 3-fluoro-2-thiophenecarboxylic acid (1 eq) in diethyl ether (0.4M) at 0° C. was added dropwise 1M Lithium Aluminum hydride in THF (1.5 eq). The mixture was stirred at 0° C. for 2 h, warmed to rt and stirred for 2 h. The reaction was quenched with water (1 mL/5 mmol of starting material) and the resulting mixture was filtered through a pad of celite eluted with diethyl ether. The filtrate was dried over MgSO₄, filtered and concentrated. The resulting crude product was used as such for next step.

Step 3: Synthesis of 2-(bromomethyl)-3-fluorothiophene

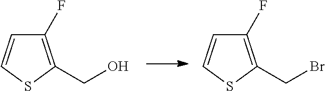

To a solution of (3-fluoro-2-thienyl)methanol (1 eq) in diethyl ether (0.6M) at 0° C. was added dropwise phosphorus tribromide (0.6 eq). The mixture was stirred at 0° C. for 1 h and poured into crushed ice. The resulting aqueous mixture was then extracted with diethyl ether (3×) the combined organic layers were washed with saturated aqueous sodium bicarbonate, dried over magnesium sulfate, filtrated and concentrated. The crude mixture was then diluted in diethyl ether (1 M) and kept at 0° C. until further use.

Step 4: Synthesis of tert-butyl 2-[(diphenylmethylene)amino]-3-(3-fluoro-2-thienyl)propanoate

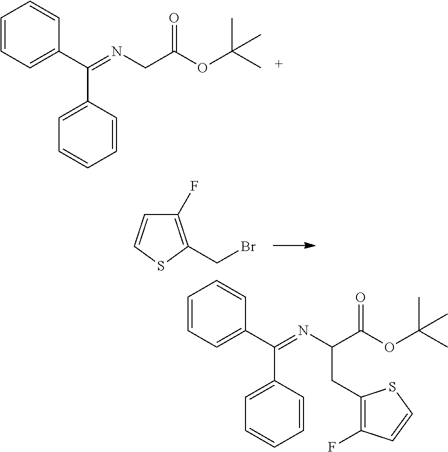

n-BuLi (1.1 eq) was added to a solution of diisopropylamine (1.2 eq) in THF (0.4M) at −40° C., DMPU (2 eq) was then added and the mixture was stirred for 15 min. The reaction mixture was then cooled to −78° C. and tert-butyl [(diphenylmethylene)amino]acetate in THF (1.5M) was added dropwise. The reaction mixture was stirred for 1 h and 2-(bromomethyl)-3-fluorothiophene (1M solution in diethyl ether) was added dropwise. The reaction mixture was stirred at −78° C. for 2 h and then warmed to rt over 2 h. Water was added and the aqueous layer was extracted with diethyl ether (3×), the combined organic layers were washed with brine, dried over magnesium sulfate, filtered and concentrated. The desired product was purified by Combiflash purification on silica gel using a gradient of 0-30% EtOAc/Hexanes.

Step 5: Synthesis of 1-carboxy-2-(3-fluoro-2-thienyl)ethanaminium chloride

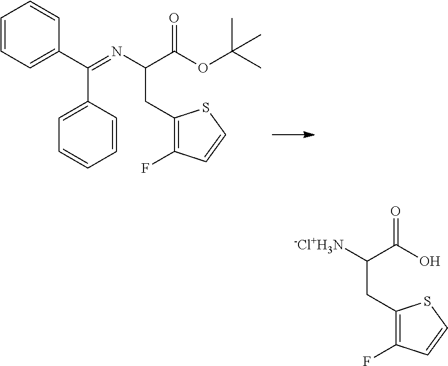

To tert-butyl 2-[(diphenylmethylene)amino]-3-(3-fluoro-2-thienyl)propanoate (1 eq) was added aqueous 6N HCl (10 eq). The mixture was stirred at reflux for 2 h and then concentrated under rotary evaporator. The residue obtained was triturated into diethyl ether to afford desired product.

Step 6: Synthesis of 2-[(tert-butoxycarbonyl)amino]-3-(3-fluoro-2-thienyl)propanoic acid

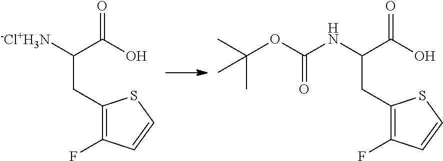

To 1-carboxy-2-(3-fluoro-2-thienyl)ethanaminium chloride (1 eq) in methanol (0.3M) were added triethylamine (3 eq) and di-tert-butyl dicarbonate (1.1 eq). The mixture was stirred at rt for 2 h, concentrated under rotary evaporator and diluted with EtOAc and 1N HCl. Organic and aqueous layers were separated and the aqueous layer was extracted with EtOAc (3×). The combined organic layers were washed with brine, dried over magnesium sulfate, filtered and concentrated to afford desired product.

Step 7: Synthesis of cis-1,5-diphenyl-6-oxa-4-azaspiro[2.4]hept-4-en-7-one

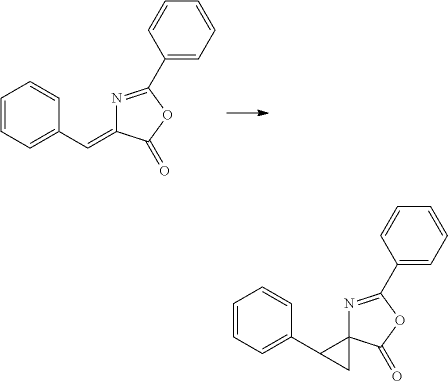

To an ice-cold solution of (4Z)-4-benzylidene-2-phenyl-1,3-oxazol-5(4H)-one (1 eq) in dichloromethane (0.3M) was added freshly prepared diazomethane (0.3M solution in diethyl ether) (10 eq). The reaction flask was covered with aluminum foil and kept in dark and the reaction mixture was warmed to rt (over 2 h) and stirred for 6 h at rt. Excess diazomethane was quenched with acetic acid and the mixture was concentrated. The residue obtained was then triturated in diethyl ether to afford desired product.

Step 8: Synthesis of cis-methyl 1-(benzoylamino)-2-phenylcyclopropanecarboxylate

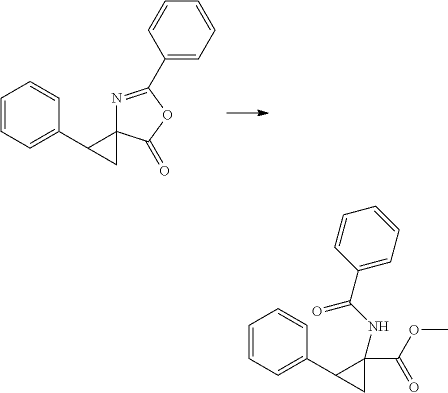

To a suspension of cis-1,5-diphenyl-6-oxa-4-azaspiro[2.4]hept-4-en-7-one (1 eq) in methanol (0.2M) was added DMAP (1.02 eq). The mixture was stirred at rt for 1.5 h and then concentrated. The residue was dissolved in CH₂Cl₂ and 1M aqueous citric acid. Organic and aqueous layers were separated and the aqueous layer was extracted with CH₂Cl₂ (3×). The combined organic layers were washed with brine, dried over sodium sulfate, filtered and concentrated to afford desired product.

Step 9: Synthesis of (1R,2R)-1-(methoxycarbonyl)-2-phenylcyclopropanaminium chloride

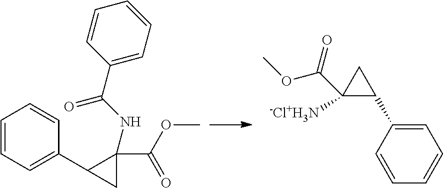

To cis methyl 1-(benzoylamino)-2-phenylcyclopropanecarboxylate (1 eq) in CH2Cl2 (0.2M) was slowly added triethyloxonium tetrafluoroborate (2 eq) (1M solution in CH2Cl2). The reaction mixture was heated to reflux for 4 h, cooled to rt, diluted with 1M aqueous KH2PO4 (2 eq). Organic and aqueous layer were separated and the aqueous layer was extracted with CH2Cl2 (3×). The combined organic layers were washed brine, dried over sodium sulfate, filtered and concentrated to afford desired product. The resulting imidate was dissolved in dry diethyl ether (1M), cooled to −20° C. and dry diethyl ether saturated with HCl was added (excess). The mixture was stirred for 20 min and 1N aqueous HCl (2 eq) was added. The reaction was allowed to return to rt and stirred for 3 h. The aqueous layer was washed with diethyl ether and concentrated under rotary evaporator. The white solid formed was triturated with diethyl ether to afford desired.

1-(methoxycarbonyl)-2-phenylcyclopropanaminium chloride was then suspended in CH₂Cl₂ and neutralized with saturated aqueous sodium bicarbonate. Organic and aqueous layers were separated and the aqueous layer was extracted with CH₂Cl₂ (3×). The combined organic layers were washed with brine, dried over sodium sulfate, filtered and concentrated. The racemic free amine was dissolved in EtOH and resolved by chiral HPLC purification with Chiralpak A/D column using 15% EtOH/Hexanes as eluant. The isomer eluting second (more polar) was dissolved in diethyl ether and treated with dry 3N HCl in diethyl ether to afford (1R,2R)-1-(methoxycarbonyl)-2-phenylcyclopropanaminium chloride.

Step 10: Synthesis of methyl(1R,2R)-1-{[2-[(tert-butoxycarbonyl)amino]-3-(3-fluoro-2-thienyl)propanoyl]amino}-2-phenylcyclopropanecarboxylate

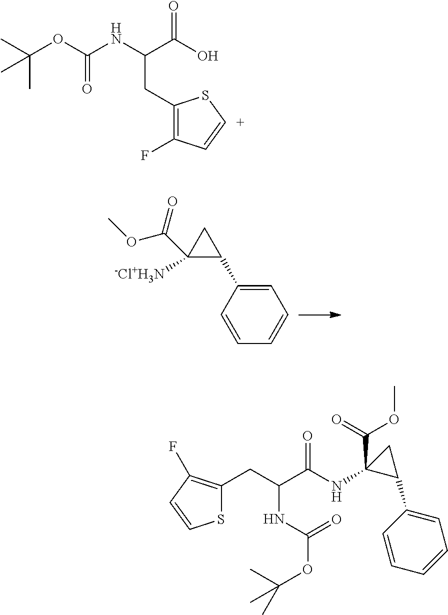

To a solution of 2-[(tert-butoxycarbonyl)amino]-3-(3-fluoro-2-thienyl)propanoic acid (1 eq) in DMF (0.2M) at 0° C. was added HATU (1.1 eq) followed by the addition of 2,6-lutidine (1.1 eq). The mixture was stirred for 15 min and (1R,2R)-1-(methoxycarbonyl)-2-phenylcyclopropanaminium chloride (1 eq) was added and followed by the addition of 2,6-lutidine (1.1 eq). The reaction was stirred for 16 h at 0° C. and then diluted in EtOAc and 1N HCl. Organic and aqueous layers were separated and the aqueous layer was extracted with EtOAc (2×). The combined organic layers were washed with brine (2×), saturated aqueous sodium bicarbonate (1×) and brine (2×), dried over magnesium sulfate, filtered and concentrated. The desired product was purified by Combiflash purification on silica gel using a gradient of 20-50% EtOAc/Hexanes.

Step 11: Synthesis of (1R,2R)-1-{[2-[(tert-butoxycarbonyl)amino]-3-(3-fluoro-2-thienyl)propanoyl]amino}-2-phenylcyclopropanecarboxylic acid

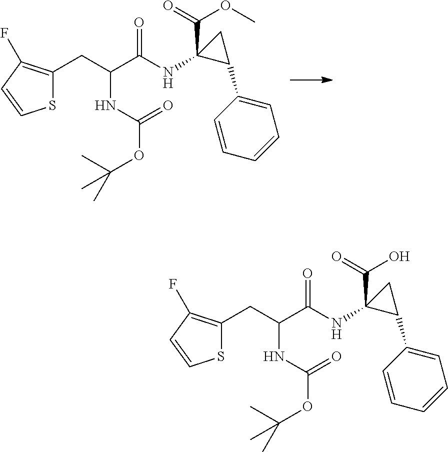

To a solution of methyl(1R,2R)-1-{[2-[(tert-butoxycarbonyl)amino]-3-(3-fluoro-2-thienyl)propanoyl]amino}-2-phenylcyclopropanecarboxylate (1 eq) in 1,4-dioxane (0.2M) was added aqueous 1N LiOH (4 eq). The mixture was stirred at rt for 4 h and then acidified to pH 2 with 1N HCl. The mixture was extracted with EtOAc (3×), the combined organic layers were dried over sodium sulfate, filtered and concentrated. The residue obtained was triturated in diethyl ether to afford desired product as a solid.

Step 12: Synthesis of tert-butyl{2-{[(1R,2R)-1-(aminocarbonyl)-2-phenylcyclopropyl]amino}-1-[(3-fluoro-2-thienyl)methyl]-2-oxoethyl}carbamate

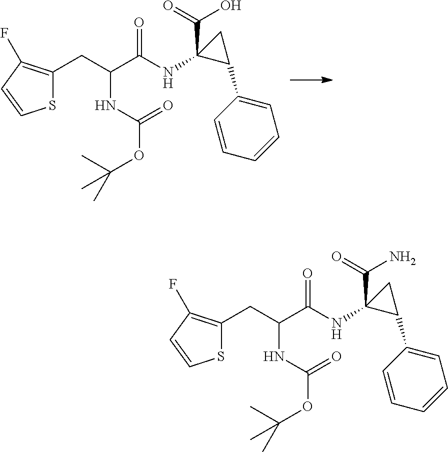

To a solution of (1R,2R)-1-{[2-[(tert-butoxycarbonyl)amino]-3-(3-fluoro-2-thienyl)propanoyl]amino}-2-phenylcyclopropanecarboxylic acid (1 eq) in DMF (0.2M) at 0° C. was added HATU (2 eq) followed by the dropwise addition of ammonium hydroxide (3 eq). The mixture was stirred at 0° C. for 3 h and then diluted with water and stirred for 10 min. The precipitate formed was filtered off, washed with water and diethyl ether and dried under high vacuum.

Step 13: Synthesis of tert-butyl {2-{[(1R,2R)-1-cyano-2-phenylcyclopropyl]amino}-1-[(3-fluoro-2-thienyl)methyl]-2-oxoethyl}carbamate

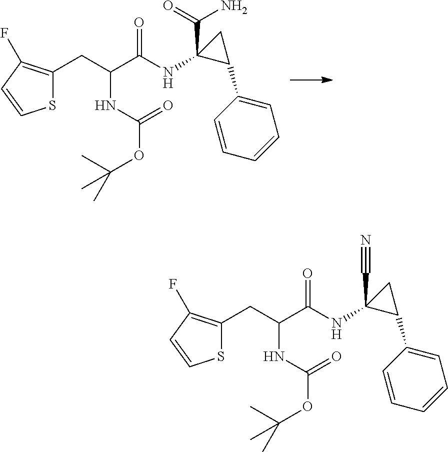

To a solution of tert-butyl{2-{[(1R,2R)-1-(aminocarbonyl)-2-phenylcyclopropyl]amino}-1-[(3-fluoro-2-thienyl)methyl]-2-oxoethyl}carbamate (1 eq) in THF (0.2M) at 0° C. was added aqueous NEt₃ (2.8 eq) followed by slow addition of trifluoroacetic anhydride (1.4 eq). The mixture was stirred at 0° C. for 1 h and aqueous saturated sodium bicarbonate was added. The mixture was extracted with EtOAc (3×), the combined organic layers were dried over sodium sulfate, filtered and concentrated. The desired product was purified by Combiflash purification on silica gel using a gradient of 20-50% EtOAc/Hexanes.

Step 14: Synthesis of (2S)-2-amino-N-[(1R,2R)-1-cyano-2-phenylcyclopropyl]-3-(3-fluoro-2-thienylpropanamide

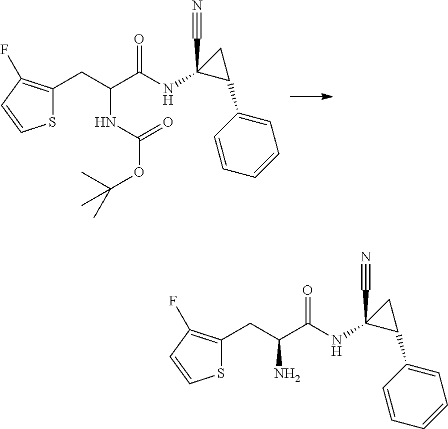

To a solution of tert-butyl{2-{[(1R,2R)-1-(aminocarbonyl)-2-phenylcyclopropyl]amino}-1-[(3-fluoro-2-thienyl)methyl]-2-oxoethyl}carbamate (1 eq) in THF (0.4M) at rt was added methanesulfonic acid (10 eq). The mixture was stirred at rt for 2 h and neutralized with aqueous saturated sodium bicarbonate. The mixture was extracted with EtOAc (3×), the combined organic layers were dried over sodium sulfate, filtered and concentrated. The diastereomeric mixture was then dissolved in ethanol and resolved by chiral HPLC purification using Chiralpak O/D column using 30% iPrOH/Hexanes as eluant. The isomer eluting first (less polar) was identified as the desired (2S)-2-amino-N-[(1R,2R)-1-cyano-2-phenylcyclopropyl]-3-(3-fluoro-2-thienylpropanamide.

M+1 (+ESI)=330.2

### Example 6

### 4′-(1-carbamoylcyclopropyl)-N-[(2S)-1-[(cyanomethyl)amino]-3-(3,5-dimethylphenyl)-1-oxopropan-2-yl]biphenyl-4-carboxamide

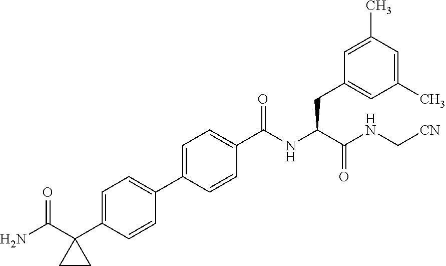

Step 1: N-(tert-butoxycarbonyl)-3,5-dimethyl-L-phenylalanine

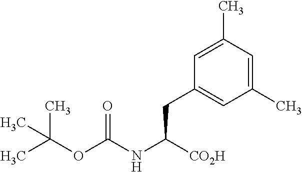

This compound was prepared in analogous manner to compound 2 of the following reference (J. Med. Chem. 2001, 44 p. 4524-4534), using 3,5-dimethylbenzyl bromide as the alkylating agent.

Step 2: Nα-(tert-butoxycarbonyl)-N-(cyanomethyl)-3,5-dimethyl-L-phenylalaninamide

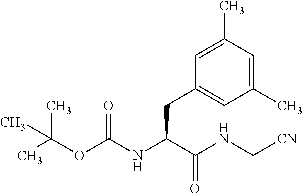

To a solution of the acid from Step 1 (7.2 g, 24.78 mmol), aminoacetonitrile hydrochloride (2.75 g, 29.7 mmol), and HATU (14.1 g, 37.2 mmol) in DMF (200 mL) was added diisopropylethylamine (13 mL, 74.3 mmol). The resulting solution was stirred overnight at room temperature. The mixture was diluted with 800 mL of water and the product was extracted twice with a 2:1 mixture of EtOAc/Et₂O. The organic layer was washed with water and brine, and was then dried (MgSO₄), filtered, and evaporated under reduced pressure. The resulting solid was stirred vigorously with 1:10 EtOAc/hexane (50 mL) for 1.5 h before filtering to give the title compound as a pale yellow solid (6.4 g).

Step 3: N-(cyanomethyl)-3,5-dimethyl-L-phenylalaninamide

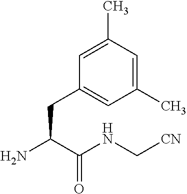

To a solution of the product from Step 2 (6.4 g, 19.3 mmol) in THF (90 mL) was added methanesulfonic acid (6.27 mL, 97 mmol). The resulting solution was stirred overnight at room temperature. The mixture was then concentrated under vacuum to remove most of the THF, and was then carefully partitioned between EtOAc (300 mL) and saturated NaHCO₃ solution (120 mL). The organic layer was washed with water and brine, and was then dried (MgSO₄), filtered, and evaporated. The resulting solid was stirred vigorously in 1:10 EtOAc/hexane (50 mL) for 1 h to give, after filtration, the title compound as a white solid (3.5 g).

### Step 4: 1-(4-Bromophenyl)cyclopropanecarboxamide

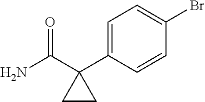

The title compound was prepared as in example 3 of WO2005056529.

Step 5: 4′-(1-Carbamoylcyclopropyl)biphenyl-4-carboxylic acid

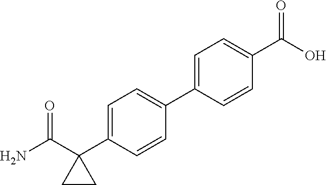

A solution of the bromide from Step 4 (1.0 g, 4.16 mmol), commercially available 4-(dihydroxyboranyl)benzoic acid (0.73 g, 4.37 mmol), sodium carbonate (2 M aq. solution, 5.2 mL, 10.4 mmol), tetrakis(triphenylphosphine)palladium(0) (20 mg, 0.017 mmol) in water (8 mL) and acetonitrile (12 mL) was heated to 60° C. and stirred overnight. The reaction mixture was cooled, filter and the mother liquors were concentrated. The residual aqueous mixture was acidified with 1 N HCl. The resultant milky suspension was filtered to provide a pale grey solid which was further dried under high vacuum to provide the title compound (1.1 g).

Step 6: 4′-(1-carbamoylcyclopropyl)-N-[(2S)-1-[(cyanomethyl)amino]-3-(3,5-dimethylphenyl)-1-oxopropan-2-yl]biphenyl-4-carboxamide

To a stirred suspension of the amine from Step 3 (420 mg, 1.82 mmol), the acid from Step 5 (500 mg, 1.78 mmol) and HATU (880 mg, 2.31 mmol) in DMF (20 mL) was added diisopropylethylamine (0.93 mL, 5.33 mmol). The resultant solution was stirred at room temperature overnight. The reaction was then slowly diluted with water (150 mL) and the resultant solid was isolated by filtration, washed with water and air dried to provide the title compound (840 mg).

¹H NMR δ (ppm) (DMSO-d₆): 8.79 (1H, t, J=5.66 Hz), 8.71 (1H, d, J=8.18 Hz), 7.92 (2H, d, J=8.16 Hz), 7.75 (4H, dd, J=25.80, 8.07 Hz), 7.46 (2H, d, J=7.99 Hz), 7.06 (1H, s), 6.95 (2H, s), 6.81 (1H, s), 6.29 (1H, s), 4.69-4.62 (1H, m), 4.17 (2H, d, J=5.50 Hz), 3.06 (1H, dd, J=13.62, 4.68 Hz), 2.98-2.88 (1H, m), 2.21 (6H, s), 1.38-1.34 (2H, m), 1.02-0.98 (2H, m).

### Example 7

### N-[(1S)-3-amino-1-cyano-3-oxopropyl]-N²-{(1S)-2,2,2-trifluoro -1-[4′-(methylsulfonyl)biphenyl-4-yl]ethyl}-L-leucinamide

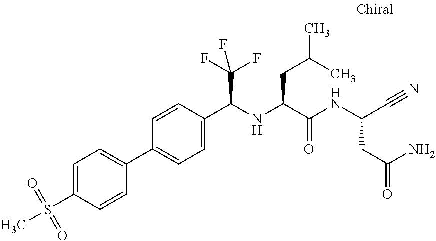

Step 1: Benzyl N²-(tert-butoxycarbonyl)-L-α-asparaginate

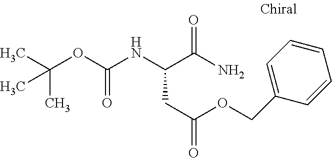

To a cold, stirred solution (5° C.) of commercially available L-aspartic acid, N-[(1,1-dimethylethoxy)carbonyl]-, 4-(phenylmethyl)ester (1.6 g, 5 mmol) and HATU (2.9 g, 7.6 mmol) in DMF (25 mL) was added dropwise ammonium hydroxide (14.8 M, 1.2 mL, 18 mmol). The resulting bright yellow suspension was stirred at room temperature overnight. The suspension was partitioned between EtOAc and saturated aqueous NaHCO₃. The organic layer was dried (Na₂SO₄) and concentrated to provide the title compound as a white solid (2 g).

Step 2: Benzyl (3S)-3-[(tert-butoxycarbonyl)amino]-3-cyanopropanoate

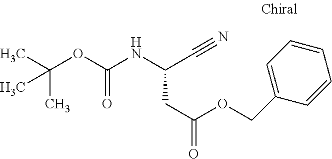

To a cold, stirred solution (0° C.) of the product from Step 1 (1.4 g, 3.5 mmol) and pyridine (1.5 mL, 18.5 mmol) in 1,4-dioxane (15 mL) was added, dropwise, trifluoroacetic anhydride (1 mL, 7 mmol). The reaction mixture was stirred at room temperature for 30 min and then partitioned between EtOAc and saturated aqueous NaHCO₃. The organic layer was dried (Na₂SO₄), concentrated and purified by chromatography (40 g Biotage cartridge eluting with 10% EtOAc/Hexanes to 30% EtOAc/Hexanes over 20 min @ 35 mL/min). The title compound was obtained as a white solid (0.9 g).

Step 3: Benzyl (3S)-3-amino-3-cyanopropanoate

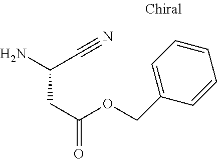

To a stirred solution of the product from Step 2 (896 mg, 2.0 mmol) in THF (8 mL) was added, dropwise over 2 minutes, methane sulfonic acid (1.3 mL, 20 mmol). The resulting solution was stirred at room temperature for 5 hours and then partitioned between EtOAc and saturated aqueous NaHCO₃. The aqueous layer was extracted 2× with EtOAc. The combined organic layers were dried (Na₂SO₄) and concentrated to provide the title compound as an amber oil (515 mg).

Step 4: N-{(1S)-2,2,2-trifluoro-1-[4′-(methylsulfonyl)biphenyl-4-yl]ethyl}-L-leucine

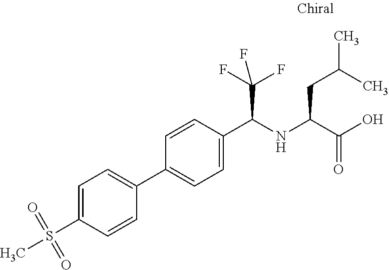

The title compound was prepared as in example 8 of WO200375836.

Step 5: Benzyl (3S)-3-cyano-3-[(N-{(1S)-2,2,2-trifluoro-1-[4′-(methylsulfonyl)biphenyl-4-yl]ethyl}-L-leucyl)amino]propanoate

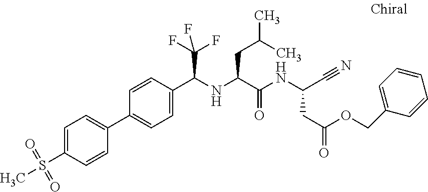

A cold solution (0° C.) of the amine from Step 3 (515 mg, 2.5 mmol) and Et₃N (1 mL, 7.2 mmol) in DMF (4 mL) was added slowly to a cold (0° C.), stirred solution of the acid from Step 4 (1.52 g, 2.4 mmol) and HATU (1.4 g, 3.7 mmol) in DMF (9 mL). The reaction mixture was then stirred at room temperature overnight and then partitioned between EtOAc and saturated aqueous NaHCO₃. The organic layer was dried (Na₂SO₄), concentrated and purified by chromatography (90 g Biotage cartridge eluting with 25% EtOAc/Hexanes to 75% EtOAc/Hexanes). The product was crystallized in EtOAc/Hexanes (1:3, 40 mL) to provide the title compound (800 mg) as a white solid. LC-MS: m/z=630.2 (MH+); mp 111° C.

Step 6: (3S)-3-cyano-3-[(N-{(1S)-2,2,2-trifluoro-1-[4′-(methylsulfonyl)biphenyl-4-yl]ethyl}-L-leucyl)amino]propanoic acid

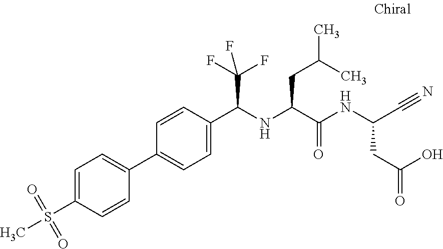

To a cold, stirred solution (0° C.) of the product from Step 5 (370 mg, 0.59 mmol) in THF (3 mL) and MeOH (1 mL) was added, dropwise, aqueous LiOH (1 N, 0.7 mL). The reaction mixture was stirred at room temperature overnight and then partitioned between EtOAc and 25% w/w aqueous NH₄OAc. The organic layer was dried (Na₂SO₄), concentrated and purified by flash column chromatography (eluting with 0% EtOH/EtOAc to 30% EtOH/EtOAc to 50% EtOH/EtOAc). The title compound was obtained as a colourless gum (170 mg). LC-MS: m/z=538 (M−H)⁺.

Step 7: N-[(1S)-3-amino-1-cyano-3-oxopropyl]-N²-{(1S)-2,2,2-trifluoro-1-[4′-(methylsulfonyl)biphenyl-4-yl]ethyl}-L-leucinamide

To a cold, stirred solution (5° C.) of the product from Step 6 (85 mg, 0.15 mmol) and HATU (100 mg, 0.26 mmol) in DMF (1 mL) was added dropwise aqueous ammonium hydroxide (14.8 M, 50 μL, 0.74 mmol). The resulting reaction mixture was stirred at room temperature overnight. The mixture was partitioned between EtOAc and saturated aqueous NaHCO₃. The organic layer was dried (Na₂SO₄), concentrated and purified by column chromatography (12 g silica cartridge, eluting with 70% EtOAc/Hexanes to 100% EtOAc/Hexanes over 10 min @ 12 mL/min) to provide the title compound as a colourless gum (25 mg). LC-MS: m/z=539 (MH+)

¹H NMR δ (ppm)(acetone-d₆): 8.2 (1H, d), 8.03 (2H, d), 7.95 (2H, d), 7.77 (2H, d), 7.65 (2H, d), 7.08 (1H, br s), 6.58 (1H, br s), 5.05 (1H, dd), 4.39-4.45 (1H, m), 3.43-3.48 (1H, m), 3.16 (3H, s), 2.71-2.78 (2H, m), 1.86-1.93 (1H, m), 1.43-1.55 (2H, m), 0.91 (6H, d).

### Example 8

### N¹-(1-cyanocyclopropyl)-N²-((1S)-1-{-4′-[(1R)-2,2-difluoro-1-hydroxyethyl]biphenyl-4-yl}-2,2,2-trifluoroethyl)-3-(methylsulfonyl)-L-alaninamide

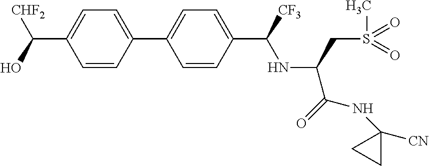

A solution of the compound of Example 1 (468 mg, 1 mmol), bis(pinacolato)diboron (279 mg, 1.1 mmol, 1.1 eq) and potassium acetate (294 mg, 3 mmol, 3 eq) in DMF (5 mL, 0.2M) was degased for 15 min using a stream of N₂ and dichloro(1,1-bis(diphenylphosphinoferrocene)palladium (II)-CH₂Cl₂ (41 mg, 0.05 mmol, 0.05 eq) was added. The mixture was warmed up to 85° C. for 3 hrs. The mixture was cooled to R.T. and 2M aqueous sodium carbonate (1.665 mL, 3.33 mmol, 3.33 eq) and (1R)-1-(4-bromophenyl)-2,2-difluoroethanol (284 mg, 1.2 mmol, 1.2 eq) were added. The mixture was degased as above and dichloro(1,1-bis(diphenylphosphinoferrocene)palladium (II)-CH₂Cl₂ (41 mg, 0.05 mmol, 0.05 eq) added.

The mixture was warmed to 85° C. for 2 hrs. It was cooled to room temp, poured into saturated NaHCO₃ and EtOAc and filtered on celite. The aqueous was separated and further extracted twice with EtOAc. Combined organic layers were washed with brine, dried and evaporated to dryness. The residue was purified by chromatography on SiO2 using 1.2:1 EtOAc and hexanes to yield a gum which was triturated in MTBE to yield the title compound (25 mg). ¹H NMR (CD₃COCD₃) δ 8.45 (1H, NH), 7.75 (4H, m), 7.6 (4H, m), 5.85-6.15 (1H, m), 5.3 (1H, OH), 4.9-5.0 (1H, m), 4.5 (1H, m), 3.8 (1H, m), 3.5-3.6 (1H, m), 3.25-3.35 (1H, m), 3.1-3.2 (4H, m), 1.35-1.45 (2H, m), 1.0-1.3 (2H, m).

### Example 9

### N²-[(1S)-1-(4-bromophenyl)-2,2,2-trifluoroethyl]-N¹-(1-cyanocyclopropyl)-S-(2,3-difluorobenzyl)-L-cysteinamide

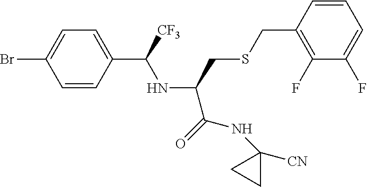

Step 1. Following the procedure of Example 1, Step 1 using 1-(bromomethyl)-2,3-difluorobenzene, methyl N-(tert-butoxycarbonyl)-S-(2,3-difluorobenzyl)-L-cysteinate was prepared and used as such in the next step.
Step 2. Following the procedure of Example 1, Step 2, using the compound of above Step 1 methyl S-(2,3-difluorobenzyl)-L-cysteinate HCl was prepared. ¹H NMR (CD₃OD) δ: 7.1-7.35 (3H, m), 4.35 (1H, m), 3.8-4.0 (5H, m), 2.95-3.13 (2H, m).
Step 3. Following the procedure of Example 1, Step 3 using the compound of above Step 2, N-[(1S)-1-(4-bromophenyl)-2,2,2-trifluoroethyl]-S-(2,3-difluorobenzyl)-L-cysteine was obtained as an impure mixture and was used as such in the next step.
Step 4. To a −5° C. solution of the compound of Step 3 (2.09 g, 4.32 mmol), HATU (2.464 g, 6.48 mmol, 1.5 eq) and 1-Amino-1-cyclopropanecarbonitrile-HCl (768 mg, 6.48 mmol, 1.5 eq) in DMF (10 mL, 0.432M) was added DIPEA (4.52 mL, 25.9 mmol, 6 eq) dropwise and the mixture was reacted at R.T. for 3 hrs. It was poured on ice and dilute HCl and extracted twice with EtOAc. The combined organic layers were washed with dilute NaHCO₃, brine, dried and the solvent was removed in vacuo. The residue was chromatographed on SiO2 using 1:2 EtOAc and hexanes to yield the title compound (1.4 g) as a mixture of isomers with about 10% of N²-[(1R)-1-(4-bromophenyl)-2,2,2-trifluoroethyl]-N¹-(1-cyanocyclopropyl)-S-(2,3-difluorobenzyl)-L-cysteinamide; this product was used as such in Example 10. MS (+ESI): 548.3, 550.2.

### Example 10

### N²-[(1S)-1-(4-bromophenyl)-2,2,2-trifluoroethyl]-N¹-(1-cyanocyclopropyl)-3-[(2,3-difluorobenzyl)-sulfonyl]-L-alaninamide

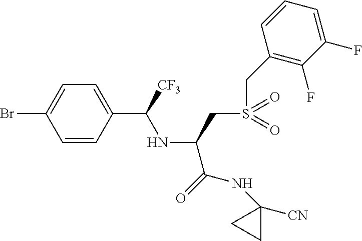

To a 0° C. suspension of the product of Example 9 (1.27 g, 2.316 mmol) in EtOAc (25 mL, 0.093M) was added tungstic acid, 99% (10 mg, 0.04 mmol, 0.017 eq) and tetrabutylammonium hydrogen sulfate (40.1 mg, 0.118 mmol, 0.051 eq). To this mixture was added hydrogen peroxide 30% (579 uL, 5.67 mmol, 2.449 eq) dropwise and the mixture was allowed to warm to RT and was stirred for 16 hrs. The mixture was washed with dilute aqueous sodium thiosulfate and brine. The organic layer was dried and the solvent was removed in vacuo. The residue was triturated in MTBE for 2 hrs and filtered to provide the title compound (1.1 g, Yield=82%). ¹H NMR (CD₃OD) δ: 8.5 (1H, NH), 7.6-7.7 (2H, d), 7.5 (2H, d), 7.25-7.45 (3H, m), 4.75-4.9 (2H, dd), 4.6 (1H, m), 3.9 (1H, m), 3.35-3.7 (3H, m), 1.4-1.5 (2H, m), 1.05-1.25 (2H, m).

### Example 11

### N²-[(1S)-1-(4-bromophenyl)-2,2,2-trifluoroethyl]-N¹-(1-cyanocyclopropyl)-3-(isobutylsulfonyl)-L-alaninamide

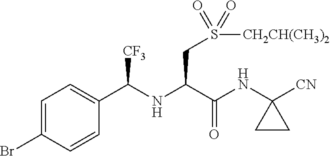

Step 1. To a suspension of L-cysteine (25 g, 206 mmol) in ethanol (200 mL, 1.03M) at 21° C. was added dropwise a solution of sodium hydroxide (16.48 g, 412 mmol, 2 eq) in ethanol (170 mL). Then 1-bromo-2-methylpropane (24.78 mL, 227 mmol, 1.102 eq) was added dropwise and the reaction mixture was stirred at 21° C. for 16 hours. It was neutralized with 2N HCl (75 mL) and concentrated to 100 mL. Then 190 mL of water was added and the pH was adjusted to 6.5 with 2N HCl. The mixture was stirred at 0° C. for 2 h to precipitate a solid which was filtered, washed with water and dried under vacuum at 50° C. to afford S-isobutyl-L-cysteine (30 g) as white powder. ¹H NMR (CD₃SOCD₃) δ: 7.70 (1H, bs), 2.98 (1H, dd), 2.72 (1H, dd), 2.43 (2H, d), 1.79-1.71 (1H, m), 0.95 (6H, d).
Step 2. Following the procedure of Example 1, Step 2, using the compound of above Step 1 methyl S-isobutyl-L-cysteinate HCl was prepared. ¹H NMR (CD₃OD) δ: 4.3 (1H, m), 3.85 (3H, s), 2.95-3.15 (2H, m), 2.5 (2H, m), 1.75-1.85 (1H, m), 1.0 (6H, d).
Step 3. Following the procedure of Example 1, Step 3 using the compound of above Step 2, N-[(1S)-1-(4-bromophenyl)-2,2,2-trifluoroethyl]-S-isobutyl-L-cysteine was obtained as an impure mixture of isomers used as such in the next step.
Step 4. Following the procedure of Example 1, Step 4, using the product of above Step 3, N²-[(1S)-1-(4-bromophenyl)-2,2,2-trifluoroethyl]-N¹-(1-cyanocyclopropyl)-S-isobutyl-L-cysteinamide was obtained as a mixture of isomers and was used as such in the next step.
Step 5. Following the procedure of Example 1, Step 5 but using the compound of the above Step 4, the title compound was obtained. ¹H NMR (CD₃COCD₃) δ: 8.45 (1H, NH), 7.65 (2H, d), 7.5 (2H, d), 4.5-4.6 (1H, m), 4.7-4.8 (1H, m), 4.4-4.5 (1H, m), 3.1-3.4 (4H, m), 2.25-2.45 (1H, m), 1.4-1.5 (2H, m), 1.05-1.3 (8H, m).

### Example 12

### N²-(1-Cyanocyclopropyl)-3-(isobutylsulfonyl)-N²-[(1S)-2,2,2-trifluoro-1-(4-fluorophenyl)ethyl]-L-alaninamide

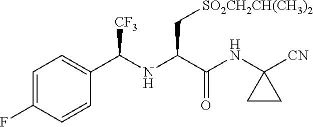

Step 1. Following the procedure of Example 1, Step 3, but using 2,2,2,4′-tetrafluoroacetophenone and methyl S-isobutyl-L-cysteinate HCl was prepared S-isobutyl-N-[(1S)-2,2,2-trifluoro-1-(4-fluorophenyl)-ethyl]-cysteine which was used as such in Step 2.
Step 2. Following the procedure of Example 1, Step 4, using the compound of above Step 1N¹-(1-cyanocyclopropyl)-S-isobutyl-N²-[(1S)-2,2,2-trifluoro-1-(4-fluorophenyl)ethyl]-L-cysteinamide was prepared and used as such in the next step.
Step 3. Following the procedure of Example 1, Step 5 using the compound of above Step 2, the title compound was prepared. MS (+ESI): 449.9 [M+1]+.
The following compounds may be prepared using the methods described above:

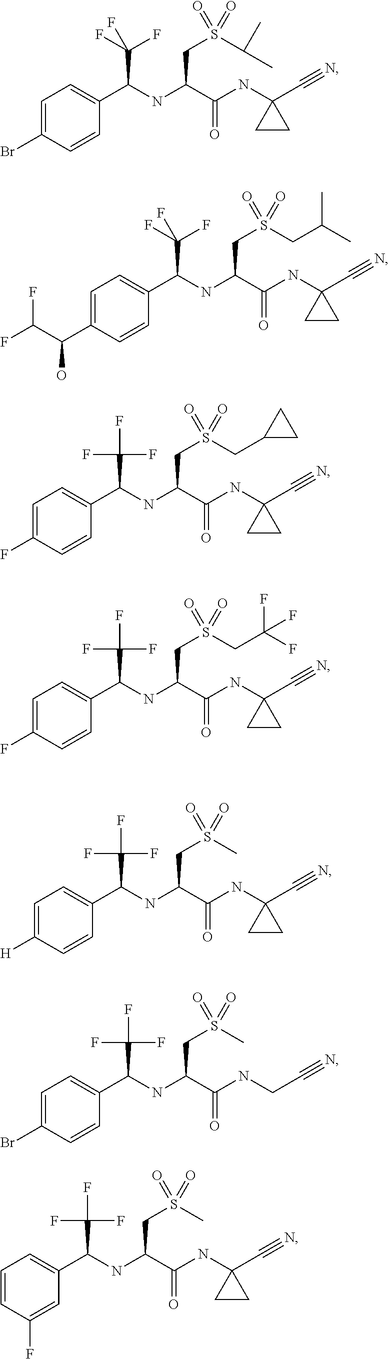

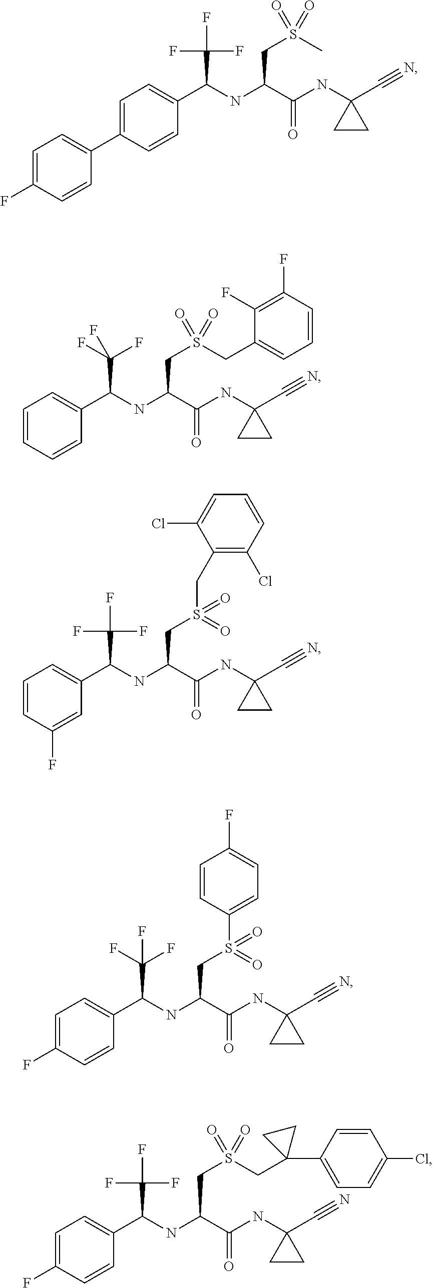

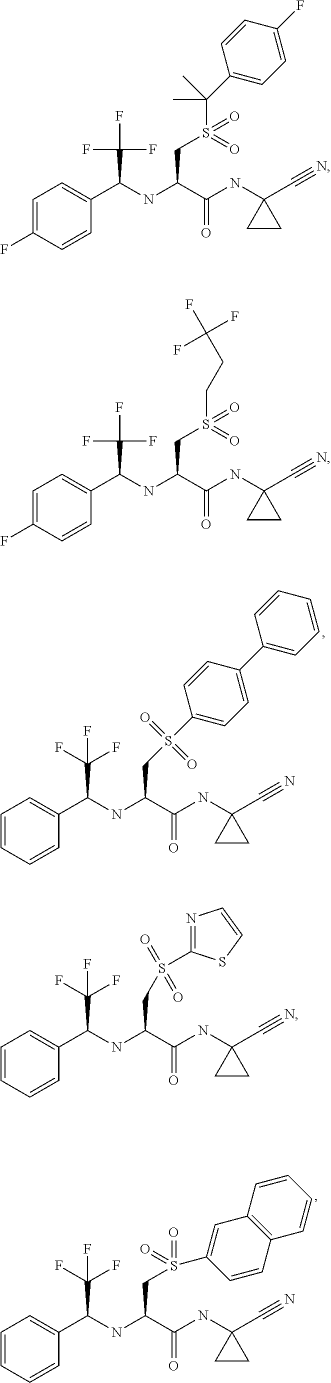

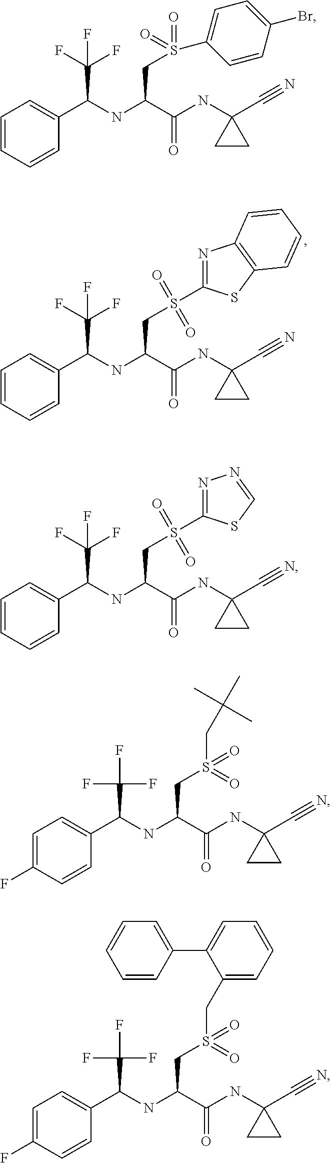

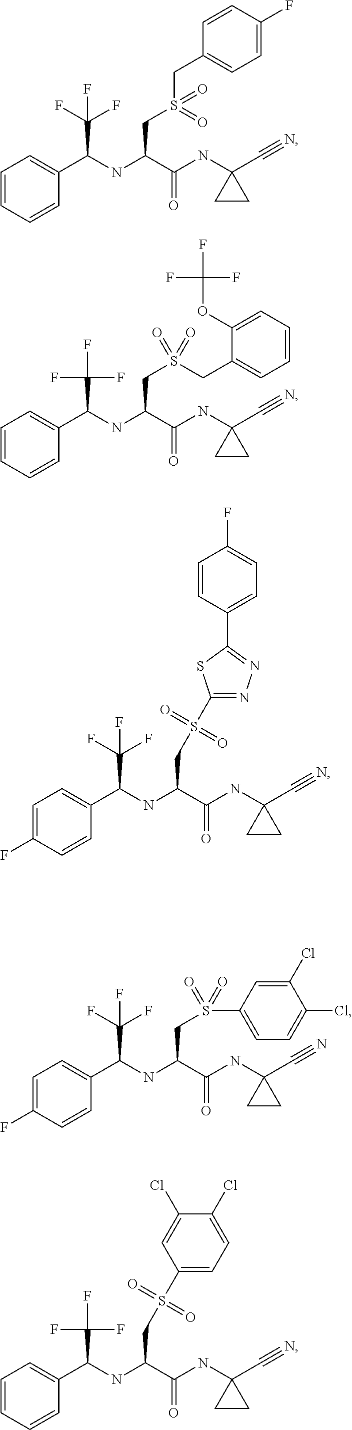

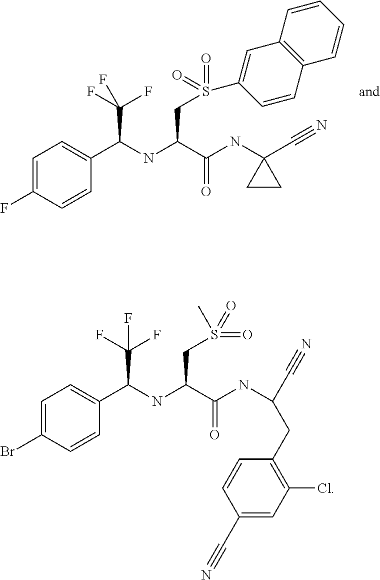

### Pharmaceutical Composition

As a specific embodiment of this invention, 100 mg of 4′-(1-carbamoylcyclopropyl)-N-[(2S)-1-[(cyanomethyl)amino]-3-(3,5-dimethylphenyl)-1-oxopropan-2-yl]biphenyl-4-carboxamide is formulated with sufficient finely divided lactose to provide a total amount of 580 to 590 mg to fill a size 0, hard-gelatin capsule.

### Biological Assays

The compounds disclosed in the present application exhibited activity in the following assays.

### Cathepsin Assay

Serial dilutions (1/3) from 500 μM down to 0.0085 μM of test compounds were prepared in dimethyl sulfoxide (DMSO). Then 2 μL of each dilution was added to 50 μL of assay buffer (MES, 50 mM (pH 5.5); EDTA, 2.5 mM; DTT, 2.5 mM and 10% DMSO) and 25 of human cathepsin protein in assay buffer solution. The assay solutions were mixed for 5-10 seconds on a shaker plate and incubated for 15 minutes at room temperature. Z-Leu-Arg-AMC (8 μM), a cathepsin substrate, in 25 μL of assay buffer was added to the assay solutions. Hydrolysis of the coumarin leaving group (AMC) from the cathepsin substrate was followed by spectrofluorometry (Exλ=355 nm; Emλ=460 nm) for 10 minutes. Percent of inhibition was calculated by fitting experimental values to standard mathematical model for dose response curve.

The cathepsin protein solutions used in the cathepsin assays were as follows:

human cathepsin K (0.4 nM);
human cathepsin L (0.5 nM);
human cathepsin B (4.0 nM); and
human cathepsin S (20 nM).

### Cruzipain Assay

Serial dilutions (1/3) from 500 μM down to 0.0085 μM of test compounds were prepared in dimethyl sulfoxide (DMSO). Then 24 of each dilution was added to 100 μL of cruzipain (500 ng/ml) in assay buffer solution (NaOAc, 50 mM (pH 5.5); DTT, 5 mM; and DMSO 10% v/v). The assay solutions were mixed for 5-10 seconds on a shaker plate and incubated for 15 minutes at room temperature. Z-Phe-Arg-AMC (20 μM) in 10 μL of assay buffer was added to the assay solutions. Hydrolysis of the coumarin leaving group (AMC) from the a cathepsin substrate was followed by spectrofluorometry (Exλ=350 nm; Emλ=460 nm) for 10 minutes. Percent of inhibition was calculated by fitting experimental values to standard mathematical model for dose response curve.

T. cruzi Epimastigotes Assay

The epimastigote form of T. cruzi (Brazilian strain) was initiated in a 25 cm² flask with a cell density of 2×10⁶ epimastigotes per ml and grown in liver infusion tryptose (LIT) broth medium, supplemented with 10% newborn calf serum (Gibco) and antibiotics, at 28° C. with agitation (80 rpm) to a cell density of 0.5×10⁷ to 1×10⁷, measured with an electronic particle counter (model ZBI; Coulter Electronics Inc., Hialeah, Fla.) and by direct counting with a hemocytometer. Various amounts of test compounds in DMSO were added to the flasks when the epimastigotes cell density reached 0.5×10⁷ to 1×10⁷ per ml then incubated for 24 to 48 h and the epimastigotes harvested during the logarithmic growth phase. The harvested epimastigotes were washed three times with 1M phosphate-buffered saline (PBS; pH 7.4) by centrifugation at 850 g for 15 minutes at 4° C. The harvested epimastigotes were reincubated in fresh LIT broth supplemented with 10% newborn calf serum and antibiotics, at 28° C. with agitation (80 rpm) and the viability of the epimistagotes was evaluated for up to one week using trypan blue exclusion (light microscopy) and [³H]-thymidine incorporation assay (see below).

T. cruzi Trypomastigote Assay

The epimastigote forms of T. cruzi were grown as described above and harvested on day 14 (stationary phase), washed three times in Grace's insect medium pH 6.5 (Invitrogen or Wisent) and induced to the trypomastigote form by metacyclogenesis with the addition of fresh Grace medium supplemented with 10% fetal calf serum (FCS) and haemin (25 μg/ml) and cultured for up to five days at 28° C. Trypomastigotes released to the supernatant were collected by a 3000 g centrifugation for 15 minutes and washed twice in Hank's balanced salt saline supplemented with 1 mM glucose (HBSS). Various amounts of test compounds in DMSO were added to the culture of trypomastigotes with a cell density of 10⁶ per ml then incubated in RPM-10% at 37° C. for 24 to 48 h. The trypomastigotes were harvested and reduction in number (parasite lysis) was determined using a Neubauer chamber and the LD₅₀ value (drug concentration that resulted in a 50% reduction in trypomastigotes when compared to an untreated control) was estimated by plotting percentage of reduction against the logarithm of drug concentration. The viability of the harvested trypomastigotes was evaluated by their ability to infect macrophages and grow in fresh media as determined by a ³H-thymidine incorporation assay (see below).

To produce more trypomastigotes the culture may be used to infect a monolayer of mammalian cells such as U937 (human macrophage), J774 (mouse macrophage) or Vero (African green monkey kidney) cells up to 4 days.

T. cruzi Amastigote Activity (Intracellular) Assay

The epimastigotes form of T. cruzi was cultured in Grace's insect medium supplemented with 10% FCS and haemin (25 μg/ml) for up to fourteen days at 28° C. to induce the formation of the metacyclic form, so that about 30% of the parasite cells were in the metacyclic form. These parasite cells were harvested and used to infect confluent mammalian cells such as U937 (human macrophage), J774 (mouse macrophage) or Vero (African green monkey kidney) cell cultures grown in 24 wells microplates in MEM at 37° C. and 5% CO₂. After the parasitic cells were allowed to infect the macrophages, the culture media was removed and various amounts of the test compounds in MEM culture medium were added to the wells and the microplates incubated for 48 h. At the end of the incubation period the media was removed and the macrogphages were fixed and stained with May Grünwald Giemsa stain. The number of amastigotes/100 macrophages (No. A/100 Mø) was determined and the anti-amastigote activity expressed as (% AA):

% AA=[1−(No. A/100 Mø)p/(No. A/100 Mø)c]×100

### ³H-thymidine Incorporation Assay

A 200 μL MEM suspension containing a mammalian cell line, such as U937 (human macrophage), J774 (mouse macrophage) or Vero (African green monkey kidney) cells, was added to each well in 96 well flat-bottom microtitre plates and incubated for 24 to 48 h at 37° C. in 5% CO₂. The medium was removed and the cells washed three times in PBS. A 200 μL mixture of MEM containing 1×10⁷/ml stationary phase T. cruzi trypomastigotes was added to each well then incubated for 24 or 48 h under the same conditions. After the incubation period, the media was removed and the cells were washed three times in PBS. The test compounds in MEM were added to the appropriate wells and incubated for up to three days. At the end of the incubation period the media was removed and the cells were washed three times in PBS. The macrophages were lysed with 0.01% sodium dodecyl sulphate (SDS) and the parasitic cells were harvested. The harvested parasitic cells were suspended in Grace's insect media and incubated at 28° C. for 48 h. At the end of the incubation period 1 μCi of ³H-thymidine in Grace's insect media was added to each well and incubated for an additional 20 h; this was harvested and ³H-thymidine incorporation was measured.

In Vitro Screening Model Against Trypanosoma b. brucei

### Parasite and Cell Cultures

The Trypanosoma brucei brucei Squib 427 strain (STIB-950: suramin-sensitive) is used. The strain is maintained by serial passage in Hirumi (HMI-9) medium, supplemented with 10% inactivated fetal calf serum. All cultures and assays are conducted at 37° C. under an atmosphere of 5% CO₂.

### Compound Solutions/Dilutions

Compound stock solutions are prepared in 100% DMSO at 20 mM or mg/mL. The compounds are serially pre-diluted (2-fold or 4-fold) in DMSO followed by a further (intermediate) dilution in demineralized water to assure a final in-test DMSO concentration of <1%.

### Drug Sensitivity Assays

Assays are performed in sterile 96-well microtiter plates, each well containing 10 uL of the compound dilutions together with 190 uL of the parasite suspension (1.5×10⁴ parasites/well). Parasite growth is compared to untreated-infected (100% parasite growth) and uninfected controls (0% growth). After 3 days incubation, parasite growth is assessed fluorimetrically after addition of 50 uL resazurin per well. After 24 hours at 37° C. fluorescence is measured (λₑₓ 550 nm, λₑₘ 590 nm). The results are expressed as % reduction in parasite growth/viability compared to infected untreated control wells and an IC₅₀ (50% inhibitory concentration) is calculated.

### Primary Screen

Trypanosoma brucei brucei Squib 427 strain (STIB-950) is used. Compounds are tested at 5 concentrations (64-16-4-1 and 0.25 uM or mg/mL). Suramin or melarsoprol are included as reference drugs. The compound is classified as inactive when the IC₅₀ is higher than 3 uM (or ug/mL). When IC₅₀ lies between 3 and 0.2 uM (or ug/mL), the compound is regarded as being moderately active. When IC₅₀ is lower than 0.2 uM (or ug/mL), the compound is classified as highly active on the condition that it also demonstrates selective action (absence of cytotoxicity). A final recommendation for activity is given after confirmatory evaluation in a secondary screening.

### Secondary Screen

Trypanosoma brucei brucei Squib 427 strain (STIB-950) is used and IC₅₀ values are determined using an extended dose range (2-fold compound dilutions). Suramin, pentamidine and melarsoprol are included as reference drugs. If indicated, T. b. rhodesiense (STIB-900) may be included as additional species. In addition, advanced selectivity evaluation is performed against a panel of unrelated organisms (bacteria, yeasts, fungi and other protozoan parasites).

In Vitro Screening Model Against Leishmania donovani and Leishmania infantum

### Parasite and Cell Cultures

Two Leishmania species (L. infantum MHOM/MA(BE)/67 and L. donovani MHOM/ET/67/L82) are used. The strains are maintained in the Golden Hamster (Mesocricetus auratus). Amastigotes are collected from the spleen of an infected donor hamster using three centrifugation purification steps (300 rpm, keeping the supernatants, 2200 rpm, keeping the supernatants and 3500 rpm, keeping the pellet) and spleen parasite burdens are assessed using the Stauber technique (see, Stauber, L. A. (1996): Characterization of strains of Leishmania donovani. Exp Parasitol. 18:1-11). Primary peritoneal mouse macrophages are used as host cell and are collected 2 days after peritoneal stimulation with a 2% potato starch suspension. All cultures and assays are conducted at 37° C. under an atmosphere os 5% CO₂.

### Compound Solutions/Dilutions

Compound stock solutions are prepared in 100% DMSO at 20 mM or mg/mL. The compounds are serially pre-diluted (2-fold or 4-fold) in DMSO followed by a further (intermediate) dilution in demineralized water to assure a final in-test DMSO concentration of <1%.

### Drug Sensitivity Assays

Assays are performed in sterile 96-well microtiter plates, each well containing 10 uL of the compound dilutions together with 190 uL of macrophage/parasite inoculum (3.10⁴ cells+4.5.10⁵ parasites/well). The inoculum is prepared in RPMI-1640 medium, supplemented with 200 mM L-glutaine, 16.5 mM NaHCO₃ and 5% inactivated fetal calf serum. Parasite multiplication is compared to untreated-infected controls (100% growth) and uninfected controls (0% growth). After 5 days incubation, parasite burdens (mean number of amastigotes/macrophage) are microscopically assessed after Giemsa staining. The results are expressed as % reduction of total parasite burden compared to untreated control wells and an IC₅₀ (50% inhibitory concentration) is calculated.

### Primary Screen

Leishmania infantum MHOM/MA(BE)/67 strain is used. Compounds are tested at 5 concentrations (64-16-4-1 and 0.25 uM or mg/mL) Sodium-stibogluconate and miltefosin are included as reference drugs. A test compound is classified as inactive when the IC₅₀ is higher than 15 uM (or ug/mL). When IC₅₀ lies between 15 and 5 uM (or ug/mL), the compound is regarded as being moderately active. If the IC₅₀ is lower than 5 uM (or ug/mL), the compound is classified as highly active on the condition that it also demonstrates selective action (absence of cytotoxicity against primary peritoneal macrophages). A final recommendation for activity is given after confirmatory evaluation in a secondary screening.

### Secondary Screen

Leishmania infantum MHOM/MA(BE)/67 and Leishmania donovani MHOM/ET/67/L82 strains are used and the IC₅₀ values are determined using an extended dose range (2-fold compound dilutions). Pentostam®, miltesfosine, fungizone and PX-6518 are included as reference drugs. Advanced selectivity evaluation is performed against a panel of unrelated organisms (bacteria, yeasts, fungi and other protozoan parasites).

1. A compound of formula I: 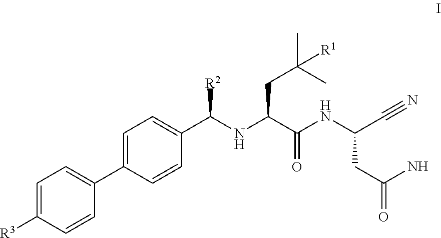
wherein R¹ is hydrogen or halo;
R² is C₁₋₃ alkyl which is substituted with two to seven halo;
R³ is hydrogen, C₁₋₆ alkyl, halo or —SOₘ(C₁₋₆ alkyl);
m is an integer from zero to two;
or a pharmaceutically acceptable salt thereof.

2. The compound of claim 1 wherein R¹ is hydrogen and R² is trifluoromethyl; or a pharmaceutically acceptable salt thereof.

3. A compound of formula II: 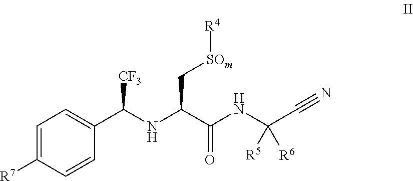
wherein R⁴ is C₁₋₆alkyl, C₁₋₆ haloalkyl, C₃₋₆cycloalkyl, aryl, (C₁₋₆alkyl)aryl, heteroaryl, or (C₁₋₆alkyl)heteroaryl, wherein said aryl and heteroaryl groups are optionally substituted with 1 to 3 substituents independently selected from the group consisting of C₁₋₆alkyl, halo, C₁₋₆ haloalkyl and cyano;
R⁵ is hydrogen, C₁₋₆ alkyl or benzyl, wherein said alkyl group is optionally substituted with 1 to 6 halo and said benzyl group is optionally substituted with one to three groups independently selected from the group consisting of halo, cyano, hydroxyl, C₁₋₆ alkyl and SOₘ;
R⁶ is hydrogen or C₁₋₆ alkyl, wherein said alkyl group is optionally substituted with 1 to 6 halo;
or R⁵ and R⁶, together with the carbon atom to which they are attached, form a C₃₋₈ cycloalkyl ring which is optionally substituted with C₁₋₆ alkyl or halo;
R⁷ is hydrogen, halo, C₁₋₆alkyl, C₁₋₆ haloalkyl, C₃₋₆cycloalkyl, aryl or heteroaryl wherein said aryl and heteroaryl groups are optionally substituted with 1 to 3 substituents independently selected from the group consisting of halo, hydroxyl, C₁₋₆alkyl, C₁₋₆ haloalkyl and cyano;
m is an integer from zero to two;
or a pharmaceutically acceptable salt thereof.

4. The compound of claim 3 wherein R⁴ is C₁₋₆alkyl, or a pharmaceutically acceptable salt thereof.

5. The compound of claim 3 wherein R⁴ is (C₁₋₆alkyl)aryl, wherein said aryl group is optionally substituted with 1 to 3 halo, or a pharmaceutically acceptable salt thereof.

6. The compound of claim 3 wherein R⁵ and R⁶, together with the carbon atom to which they are attached, form a cyclopropyl ring; R⁷ is hydrogen or halo; or a pharmaceutically acceptable salt thereof.

7. A method of treating a cysteine protease-related disease in a mammal in need thereof with a therapeutically effective amount of a compound of claim 3.

8. The method according to claim 7 wherein the cysteine protease related disease is a a parasitic disease selected from the group consisting of toxoplasmosis, malaria, African trypanosomiasis, Chagas disease, leishmaniasis, coccidiosis, giardiosis, cryptosporidiosis and schistosomiasis.

9. The method according to claim 8 further comprising another agent selected from the group consisting of: nifurtimox, benznidazole, allopurinol, terbinafine, lovastatin, ketoconazole, itraconazole, posaconazole, miltefosine, ilmofosine, pamidronate, alendronate, risedronate, chloroquine, proguanil, mefloquine, quinine, pyrimethamine-sulphadoxine, doxocycline, berberine, halofantrine, primaquine, atovaquone, pyrimethamine-dapsone, artemisinin, quinhaosu. meglumine antimonite, sodium stibogluconate, amphotericin B, praziquantel, oxamniquine, pentamidine, melarsoprol, suramin and eflornithine and the pharmaceutically acceptable salts and mixtures thereof.
